(19) United States
(12) Patent Application Publication
(10) Pub. No.: US 2008/0176810 A1
(43) Pub. Date: Jul. 24, 2008

(54) Use of 3, 4', 5-Trihydroxy-Stilbene-3-Beta-D-glucoside in Prepartion of Medicines For Treating and/or Preventing Ischemic Heart Disease

(75) Inventors: Jinhua Zhao, Guangdong (CN); Jiangping Xu, Guangdong (CN); Hui Kang, Guangdong (CN); Bing Wang, Guangdong (CN); Jing Li, Guangdong (CN); Hanlin Feng, Guangdong (CN); Lin Yu, Guangdong (CN)

Correspondence Address: BARNES & THORNBURG LLP, P.O. BOX 2786, CHICAGO, IL, 60690-2786, US

(73) Assignee: SHENZHEN NEPTUNUS PHARMACEUTICAL CO., LTD., Shenzhen, Guangdong (CN)

(21) Appl. No.: 11/720,451

(22) PCT Filed: Nov. 14, 2005
(86) PCT No.: PCT/CN05/01912

§ 371 Date: Aug. 13, 2007

(30) Foreign Application Priority Data
Dec. 1, 2004 (CN) 200410052470.9

(51) Int. Cl. A61K 31/7028; A61P 9/10

(52) U.S. Cl. 514/27

(57) ABSTRACT

The present invention provides new use of 3,4′,5-trihydroxy-stilbene-3-β-D-glucoside. 3,4′,5-trihydroxy-stilbene-3-β-D-glucoside has efficacies of anti-myocardial ischemia by intravenous injection and/or oral administration. It is advantageous that 3,4′,5-trihydroxy-stilbene-3-β-D-glucoside as anti-myocardial ischemia drug is used to prepare the medicine for treating and/or preventing ischemic heart disease.

### TECHNICAL FIELD OF THE INVENTION

The present invention involves new use of 3,4′,5-trihydroxy-stilbene-3-β-D-glucoside, particularly, it involves the application of the compound in preparation of medicines for treating and/or preventing ischemic heart disease.

### BACKGROUND OF THE INVENTION

The structure of compound of 3,4′,5-trihydroxy-stilbene-3-β-D-glucoside, (TSG), or polydatin or peicin, is shown as formula (I):

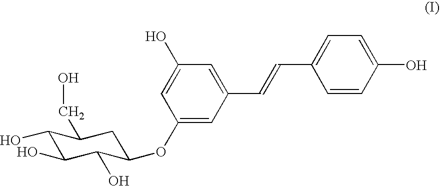

In 1960's, 3,4′,5-trihydroxy-stilbene-3-β-D-glucoside was firstly discovered in medical plant Polygonum cuspidatum Sieb, et Zucc. and later in Sakhalin spruce, grape, peanut and other plants. After 1970's, pharmacological studies of the compound of the formula (I) indicated that it has the following actions:

(1) Blood Lipid-lowering effects: for example, sublingual administration at the dose of 2.2 mg/kg/d in hyperlipemia patients might reduce the ratio of total cholesterol general cholesterin to high density lipoprotein, and low density lipoprotein to high density lipoprotein (Zhang Peiwen et al. Journal of First Military Medical University 1995; 15(1): 47-48).

(2) Inhibition of platelet aggregation and thrombogenesis: For example, 6˜100 μmol/L TSG inhibited platelet aggregation and production of thromboxane B2 induced by arachidonic acid, adenosine diphosphate and adrenalin (Shan Chunwen et al. Acta Pharmacologica Sinica, 1990, 11(6): 527˜530);

(3) Positive inotropic effect: It can enhance the contractive amplitude and frequency of cultured cardiac myocyte (Jin Chunhua et al. Chinese Pharmacological Bulletin, 2000; 16 (4): 400˜402);

(4) Antioxidation: TSG has an effect of inhibiting or scavenging free radicals produced by PMNs respiratory burst, xanthine system and VitC- Cu²⁺ system (Tian Jingwei et al. Chinese Traditional and Herbal drugs, 2001, 32 (10): 918˜920);

(5) Effect on improving microcirculation and treatment shock (Zhao kesen, Jin Lijuan; Shock and Its Molecular Basis, Science Press, Beijing, 1989; 319˜323).

Ischemic heart disease is a kind of heart disease caused by stenosis or block of blood vessel as a result of coronary atherosclerosis, or/and heart disease induced by ischemia, anoxia or necrosis resulted from functional change (spasm) of coronary artery, collectively termed as coronary atherosclerotic heart disease, or CHD). CHD is a common disease that imperils people's health seriously. CHD may be classified as asymptomatic myocardial ischemia, angina cordis, myocardial infarction, ischemic cardiomyopathy, heart failure and sudden death.

Treatment of CHD includes medical therapy, interventional therapy and surgical therapy. At present, most commonly used antimyocardial ischemic medicine in clinic includes nitrate esters, β receptor blocker and calcium antagonist. Other myocardial ischemic antagonist includes angiotesin-converting enzyme inhibitor, specific bradycardic agent and so on. The common features of such medicine are decreasing cardiac work load and reducing myocardial oxygen consumption by vasodilation so as to alleviate the symptoms of CHD.

Nitrovasodilators, or nitrate esters, may release NO, and it relaxes vascular smooth muscle by raising cGMP. Nitrate esters used in clinic, like nitroglycerin and isosorbide dinitrate, may usually alleviate angina cordis of all kinds immediately, and are widely used in prevention and treatment of angina cordis. However, “nitrate estersis only a kind of medicine that may alleviate symptoms but there is no evidence to indicate it is beneficial to outcome of illness. Theoretically, such medicine may reflectively accelerate heart rate and might have negative effect on long-term outcome of myocardial ischemia (Hu Dayi et al. Evidence-based Cardiovascology, Tianjin Science and Technology Press 2001). Additionally, nitrate esters medicine has the adverse effect of increasing intracranial pressure and inducing glaucoma, and it may also produce drug resistance soon.

Application of calcium antagonist and β receptor blocker in cardiovascular medical research was of milestone significance. So far, such medicine is still one of the most popular medicines in treatment of CHD. Calcium antagonist like nifedipine has the pharmacological effects of inhibiting myocardial contractility, reducing myocardial oxygen consumption; releasing coronary artery spasm, improving blood supply to myocardium; expanding surrounding blood vessels and mitigating heart load. But more than two decades after clinical application of the first generation of calcium antagonist, it was found such medicine might increase the risk of myocardial infarction. Clinical test suggested calcium antagonist had no improvement to prognosis of deformed myocardial infarction, varied angina cordis and cardiac insufficiency.” (Su Dingfeng et al. Cardiovascular Pharmacology, Science Press, 2001).

Research results suggested conventional anti-myocardial ischemia medicine (nitrate esters, β receptor blocker and calcium antagonist) may alleviate angina cordis but its effect on prognosis of exertional angina pectoris is unknown. At present there is no information on effect of anti-myocardial ischemia medicine on outcome of unstable angina pectoris. Short-acting nifedipine may increase mortality of acute coronary artery syndrome, while angiotensin and startin that can reduce blood lipid have indirect effect of anti-myocarial ischemia. (Hu Dayi et al. Evidence-based Cardiovascology, Tianjin Science and Technology Press 2001).

Apparently, there are many medicines available for clinical treatment of CHD, however, new medicines which are safer and more effective treatment of CHD are highly in demand to better enhance prognosis and outcome of myocardial ischemic heart disease.

As stated above, the existing literature reported the cardiovascular activity of 3,4′,5-trihydroxy-stilbene-3-β-D-glucoside. but its applicable value is shown mainly in its anti-shock effect and activity of blood lipid-lowering. No references of this compound involved or suggested its anti-myocardial ischemic effect and potential value of use in treatment and/or prevention of CHD. The reasons may be those:

The cause of CHD is the reduction of blood supply to myocardium due to stenosis of coronal artery, leading to contradiction of demand and supply of blood to cardiac muscles and a series of symptoms like pain. Generally, most antimyocarial ischemic medicines focus on improvement of hemodynamical characters of the patient, in particular the reduction of heart load and myocardial oxygen consumption in the end. Most frequently used medicines for treatment of CHD in clinic such as nitrate ester, β receptor blocker and calcium antagonist medicines as stated above have such characters in effect.

According to the existing literature, 3,4′,5-trihydroxy-stilbene-3-β-D-glucoside may enhance the contractive amplitude and frequency of cultured myocardium in vitro and isolated heart, and increase intracellular calcium concentration of myocardium (Jin Chunhua, et al. Chinese Pharmacological Bulletin, 2000, 16(4): 400˜402; Jin Xingzhong, et al. Journal of First Military Medical University, 1992; 12(1): 31˜33); Obviously, the positive inotropic effect and positive chronotropic effect of such compound indicate it may lead to increase of heart rate and myocardial contraction strength of the patient, which definitely increases his myocardial oxygen consumption and myocardial burden, aggravating the contradiction of supply and demand for blood to cardiac muscles. Therefore, it is difficult to assess 3,4′,5-trihydroxy-stilbene-3-β-D-glucoside has the therapeutic value for CHD on basis of the existing literature, on the contrary, this compound may seemingly aggravate the contradiction of supply and demand for blood to cardiac muscles.

In the reports of effect of 3,4′,5-trihydroxy-stilbene-3-β-D-glucoside on blood vessel in the existing literature, on the one hand, it is reported 5.12 mM of 3,4′,5-trihydroxy-stilbene-3-β-D-glucoside may relax isolated carotid and pulmonary arteries of rabbits, while there is no noticeable effect at 3.28 mM. It may have antagonistic effect on vasoconstriction of noadrenalin at a concentration of 1.71 mM, while there is no noticeable effect at a concentration of 1.37 mM. (Luo Sufang et al., Journal of First Military Medical University, 1992; 12(1): 10˜13). On the other hand, this compound may increase intracellular calcium concentration in vascular smooth muscle remarkably at the concentration of 0.02 mM˜2 mM. Thus the direct effect on vascular smooth muscle is positive inotropic effect and increment of angiotasis (Jin Chunhua et al. China Pathophysiological Journal, 1998, 14(2): 195˜198; Jin Chunhua et al. Chinese Pharmacological Bulletin, 2000, 16(2): 151˜154). Apparently, it is not easy to realize the plasma drug concentration of 5.12 mM in routine administration; the pharmacokinetic study of the present invention in dogs suggested the transient maximum plasma drug concentration was about 100 μg/ml after an administration of 30 mg/kg, approximately 0.25 mM. Accordingly, a plasma drug concentration of 5.12 mM requires an administration of 600 mg/kg. Based on the research result of the present invention, such administration has come to the LD50 of animals. Comparatively speaking, the plasma drug concentration of 0.02 mM˜2 mM is what the normal administration could reach. Therefore, based on the existing literature, in normal administration, the effect of 3,4′,5-trihydroxy-stilbene-3-β-D-glucoside on blood vessels may be the enhancement of angiotasis. If it is the case, heart load should be increased thus it is not helpful to alleviate the symptoms of CHD.

Based on the existing literature, in view of the effect of hemodynamics it is difficult to judge whether 3,4′,5-trihydroxy-stilbene-3-β-D-glucoside has beneficial therapeutic effect on ischemic heart disease. On the contrary, an opposite conclusion may be drawn according to conventional speculation. Therefore, it is not difficult to understand why there is so far no report on anti-myocardial ischemic test and study of this compound.

Additionally, according to other literature, 3,4′,5-trihydroxy-stilbene-3-β-D-glucoside has protective effect on cultured myocardial cells in vitro against the injury caused by Chlorpromazine, endotoxin and others factors (Luo Sufang et al. Chinese Pharmacology Journal, 1990, 11(2): 147˜150; Zhao Qing et al. Journal of First Military Medical University, 2003, 3 (4): 364˜365). As such studies are related to chemical injury of myocardial cells cultured in vitro, and different from the pathological process and therapeutic mechanism of CHD, therefore, such studies are not related to the therapeutic value of 3,4′,5-trihydroxy-stilbene-3-β-D-glucoside to CHD as stated in the present invention.

Further, the patent application of No. CN 02134928.2 discloses a pharmaceutical composition containing polydatin or its pharmaceutically acceptable salt that may improve microcirculation, and the use thereof in preparation for medicine that may improve microcirculation. Such patent involves therapy of cardio-cerebrovascular disease of microcirculation disturbance. As ischemic heart disease is a coronary arterial occlusive disease, rather than microcirculational disturbance of capillary vessels, therefore, this patent application has nothing to do with the treatment of ischemic heart disease.

Patent application of No. CN 02139335.4 states this compound may expand coronary artery and increase its blood flow when discussing that polydatin may reduce pulmonary artery hypertension. As the examples listed in the patent application only suggest the effect of this compound on animal pulmonary pressure and hematological active factors (hematologic factors such as TXA2, PGI2 that effect coagulation) in hypoxia and ischemic animal models. But it does not involve the effect on coronary artery and its blood flow, and up date. There is no report on any research on this compound to expand coronary artery and increase its blood flow, thus, the expansion of coronary artery and increase of coronary artery blood flow stated in that patent might be only a conjecture rather than a scientific research result.

### SUMMARY OF THE INVENTION

One object of the present invention is to provide a use of the compound of 3,4′,5-trihydroxy-stilbene-3-β-D-glucoside in preparation for pharmaceutical to treat and/or prevent ischemic heart diseases.

Another object of the present invention is to provide a use of the pharmaceutical composition containing 3,4′,5-trihydroxy-stilbene-3-β-D-glucoside in preparation for pharmaceutical to treat and/or prevent ischemic heart diseases.

According to one aspect of the present invention, the ischemic heart disease as stated in the present invention include asymptomatic myocardial ischemia, angina cordis, myocardial infarction, ischemic cardiomyopathy, heart failure and sudden death. When applied to therapy or prevention of ischemic heart disease, the administrative dose of 3,4′, 5-trihydroxy-stilbene-3-β-D-glucoside (TSG) for human is 20˜300 mg/60 kg weight/time converted on body surface area based on effective therapeutic administrative dose of 2 mg˜30 mg/kg weight/time of animal (rat) in vivo test. The preferred administration range to human is 50˜200 mg/60 kg weight/time in response to weight administration of 5˜20 mg/kg for rat. TSG may be given by oral administration or intravenous injection in treatment.

According to another aspect of the present invention, the pharmaceutical composition of the present invention may be prepared by the conventional process in the art with effective ingredients of 3,4′,5-trihydroxy-stilbene-3-β-D-glucoside and pharmaceutically acceptable adjuvant. Thus the invention also involves the use of such pharmaceutical composition. When making the pharmaceutical composition of the present invention into medical preparations, the dosage forms may be as follows: preparations of oral administration such as tablet, capsule (including hard capsule, soft capsule, enteric coated capsule and micro-capsule), powder, granule and syrup; preparations of non-oral administration including injection, suppository, pill, gels and patch. In addition to these conventional forms, instant solid preparation for oral administration (like tablet and granule) and sustained release preparation for oral or non-oral administration (tablet, granule, fine granule, pill, capsule, syrup, emulsion, suspension, solution) may also be used in the present invention. The preparation in the present invention may be in the coated form or without coating, as the case may be. The most preferred dosage forms of the present invention is to apply TSG to preparations for oral and intravenous administration.

Pharmaceutically acceptable adjuvant of the present invention includes excipient, lubricant, binding agent, disintegrant, stabilizer, forming agent, coating agent and others for solid preparation, or solution, solubilizer, suspending agent, isotonizing agent, buffer, emollient, emulsifier and so on for semi-solid and liquid preparations. Furthermore, other medical additives like preservative, antioxidant, colorant, sweetener and condiment may also be used when it is necessary.

In preparation of the pharmaceutical composition, the content of effective ingredient of TSG in each preparation unit of the composition is 20 mg˜300 mg with preferred content as 50 mg˜200 mg. The preparation unit refers to the total preparation amount required for one administration. Accordingly, the TSG content refers to the total amount of TSG in the medicine in a single administration. Those skilled in this art may determine TSG content in unit preparation (each tablet or each piece of preparation) according to the requirements of the preparation and its application. For instance, for tablet, each unit preparation may be made with a content of TSG 2˜30 mg according to need of administration, 1˜10 tablets for each time of administration.

In a series of test studies of the present invention, it is justified TSG has remarkable protective effect against animal myocardial ischemia caused by a number of factors.

In one embodiment of the present invention, the effect of 3,4′,5-trihydroxy-stilbene-3-β-D-glucoside on rat myocardial ischemia induced by hypophysin was observed. Quickly injected 6 U/kg hypophysin via lingual vein may lead to epicardial and endocardial ischemia of myocardium in rats. It was characterized by quick ST segment elevation on rat ECG, which dropped down gradually from its peak about 15˜30 seconds later, lowering or inverse of T wave, or characterized by remarkable suppression of ST segment, frequent ventricular premature, and also high or complete atrioventricular block. After administration of 5, 10, 20 mg/kg for 3 consecutive days, it was found the administration of 10 mg/kg and 20 mg/kg of 3,4′,5-trihydroxy-stilbene-3-β-D-glucoside could remarkably reduce ST segment elevation on ECG resulted from myocardial injury caused by intravenous injection of hypophysin in anesthetized SD rats (compared with control group, ΔST had significant or great significant difference), suggesting 10 mg/kg and 20 mg/kg po of this compound might effectively prevent myocardial ischemic injury caused by hypophysin in rats.

In another embodiment of the present invention, the effect of intravenous injection of 3,4′,5-trihydroxy-stilbene-3-β-D-glucoside on myocardial ischemia reperfusion injury in SD rats was observed. Coronary artery ligation was used to prepare myocardial ischemia reperfusion model in the test. The doses of intravenous injection of 3,4′,5-trihydroxy-stilbene-3-β-D-glucoside used in the test was 7.5, 15, 30 mg/kg (in which, according to calculation of body surface area, dog 7.5 mg/kg equal to human administration 100 mg/person for a person weighing 70 kg) respectively. The test result suggests after myocardial ischemic reperfusion, level of serum LDH and CK increased remarkably and weight of infarct area of myocardium increased noticeably, showing an obvious difference (P<0.01) when compared with sham operation control group. Low, middle and high doses (7.5, 15, 30 mg/kg) of 3,4′,5-trihydroxy-stilbene-3-β-D-glucoside might remarkably reduce the activity of serum CK, lowering the weight of myocardial infarct area (in comparison with model group, P<0.05). Middle and high doses (15 and 30 mg/kg) of 3,4′,5-trihydroxy-stilbene-3-β-D-glucoside might remarkably reduce the level of serum LDH, (when compared with model group, P<0.05). This result indicates intravenous infusion of 3,4′,5-trihydroxy-stilbene-3-β-D-Glucoside has an protective effect against myocardial injury caused by ischemia reperfusion in rats, and it can remarkably inhibit the leakage of LDH and CK, when rat myocardium is injured due to ischemic reperfusion, and reduce the level of serum LDH and CK, lower the weight of myocardial infarction area. The effect shows the relationship of dose dependence.

In another embodiment of the present invention, therapeutic effect of 3,4′,5-trihydroxy-stilbene-3-β-D-glucoside on myocardial infarction model resulted from coronary artery ligation in dog was observed. Coronary artery ligation was also used to prepare myocardial ischemic reperfusion model in this test. The doses of 2.5, 5 and 10 mg/kg of 3,4′, 5-trihydroxy-stilbene-3-β-D-glucoside were used respectively by intravenous injection in the test. Test results indicate 5 minutes after coronary artery ligation, the sum of ST segment (ΣST) of epicardial electrogram in solvent control group was obviously elevated. At the time of 30 min it reached the peak (ΣST raised up to 18.2%) and then dropped slowly afterward. Intravenous administration of TSG at the dose of 2.5˜10 mg/kg can dose-dependently inhibit the rise of ΣST. There was significant difference between 2.5 mg/kg dose group and solvent control group at the time points of 30˜90 min. 5 mg/kg dose group had also great difference at all time points in comparison with solvent control group. As for high dose group of 10 mg/kg, obvious effect was produced immediately after administration and lasted for 120 min. The test results indicate the intravenous injection of TSG at 2.5, 5 and 10 mg/kg has obvious therapeutic effects on extent of canine acute myocardial ischemia induced by coronary artery ligation.

Similarly, TSG may dose-dependently reduce the area of myocardial ischemia and the value of N-ST on epicardium electrogram and the time of such effect can last for 120 min. In comparison of the rates of N-ST reduction at all time points in 2.5 mg/kg dose group and those in solvent control group at the same time points, there was significant difference from 30˜90 min (P<0.05), and there was also significant difference from 15˜120 min in the rates of N-ST reduction in 5 mg/kg dose group at all time points when compared with those of solvent control group at the same time points. In 10 mg/kg dose group, there was very significant difference in reduction of myocardial ischemia within the time of 5˜120 min in comparison with solvent control group.

The quantitative histological detection of the area of myocardial infarction is to adopt N-BT staining to demonstrate intravenous administration of TSG at the dose of 2.5˜10 mg/kg which can dose-dependently reduce the area of myocardial infarction. The results are consistent with those detected with epcardial electrogram. After administration of TSG 2.5, 5, 10 mg/kg, the ratio of infarct zone/left ventricle decreased very significantly when compared with that in control group (P<0.01); the ratio of infarct zone/whole heart also decreased significantly in comparison with that in control group (P<0.05˜0.01).

After coronary artery ligation, both level of serum lactate dehydrogenase (LDH) and creatine kinase (CK) raised in all groups. Administration of TSG can reduce the degree of serum LDH and CK increment significantly at doses of at 2.5, 5, 10 mg/kg, among which high dose (10 mg/kg) demonstrated a strongest pharmacodynamic action.

These test results indicate intravenous infusion of 3,4′,5-trihydroxy-stilbene-3-β-D-glucoside can reduce the extent of canine myocardial ischemia induced by coronary artery ligation and minimize the area of myocardial ischemia. The strength of such action is dose dependent. The results of quantitative detection of histology are consistent with those detected in epicardial electrogram, suggesting that TSG intravenous injection has obvious therapeutic effects on canine myocardial infarction induced by coronary artery ligation. The results of the experiment also demonstrate 3,4′,5-trihydroxy-stilbene-3-β-D-glucoside can significantly reduce the degree of serum increment of lactate dehydrogenase and creatine kinase, indicating that it can diminish the efflux of serum LDH and CK so as to minimize the cellular damage resulted from myocardial ischemia and has protective effect on myocardial cells.

In another embodiment of the present invention, protective effect on canine acute myocardial infarction of 3,4′,5-trihydroxy-stilbene-3-β-D-glucoside by intragastric administration was observed. Test model was the same as Example 2. Oral administration (intragastric administration) of 3,4′,5-trihydroxy-stilbene-3-β-D-glucoside was adopted at doses of 7.5, 15 and 30 mg/kg respectively. Test results demonstrate TSG 7.5, 15 and 30 mg/kg po might reduce the rise of ST segment induced by myocardial ischemia, with best result of 20 mg/kg group. TSG 7.5, 15 and 30 mg/kg po might reduce Σ-ST with different degree. During most period of time after intragastric perfusion, ST segments showed noticeable suppression. After administration of TSG at doses of 7.5, 15 and 30 mg/kg, N-ST dropped gradually. It showed a remarkable drop at 90 min after administration when compared with solvent control group at the same time period. TSG 10 and 20 mg/kg po might reduce the activity of serum LDH significantly. The quantitative histological detection (N-BT dyeing method) indicated TSG 10 and 20 mg/kg po may minimize the myocardial infarction area remarkably. Example 4 demonstrated oral administration of TSG has the effect to alleviate myocardial ischemia; and has a protective effect against myocardial injury induced by acute myocardial infarction in anesthetized dogs.

In another embodiment of the present invention, the effect of intravenous injection of 3,4′,5-trihydroxy-stilbene-3-β-D-glucoside on myocardial consumption of oxygen of healthy anesthetized dog was observed. The test results indicate: there was no significant difference (P>0.05) in myocardial consumption of oxygen when compared with solvent control group after intravenous injection of 3,4′,5-trihydroxy-stilbene-3-β-D-glucoside (TSG) at the doses of 2.5, 5, 10 mg/kg. Myocardial oxygen uptake rate was reduced significantly. When compared with the control group at the same time points, the drop rate was significant at 15, 60 and 90 min in 2.5 mg/kg dose group. Both 5 and 10 mg/kg groups showed remarkable drop from 15 min to 120 min. Intravenous injection of TSG at doses of 2.5, 5, 10 mg/kg might increase coronary artery flow, and the increase rate of various dose groups showed very significant difference within 15˜120 min, when compared with solvent control group at the same time points. In 120 min of TSG administration of 5, 10 mg/kg, there was very significant difference in coronary resistance drop rate at the same time points in comparison with solvent control group. TSG might increase canine cardiac output. When compared with solvent control group at the same time points, there was significant difference in cardiac output change rate in 5 mg/kg dose group at 60 and 90 min, and in 10 mg/kg dose group at 15 min and 30 min (P<0.05), and there was very significant difference at 60 and 90 min (P<0.01). The above results demonstrate 3,4′,5-trihydroxy-stilbene-3-β-D-glucoside may increase coronary artery flow and cardiac output, reduce significantly myocardial oxygen uptake rate and reduce coronary resistance in anesthetized dogs.

Effects of intravenous injection of 3,4′,5-trihydroxy-stilbene-3-β-D-glucoside on hemodynamic indexes such as heart rate, blood pressure, systolic and diastolic function of heart in anesthetized normal dogs were observed in another embodiment of the present invention. Test results demonstrate after TSG 2.5, 5 mg/kg iv, there was variation in heart rate, blood pressure, left ventricular pressure and the maximal rate of left intraventricular pressure changes. But there was no obvious difference when compared with that before administration. TSG 10 mg/kg iv raised blood pressure of anesthetized dog. But there was no significant difference when compared with that before administration, and there were no significant change in other indexes.

Therapeutic effect of intragastric administration of 3,4′,5-trihydroxy-stilbene-3-β-D-glucoside (TSG) on chronic myocardial ischemia in rat was observed in another embodiment of the present invention. Test model of chronic myocardial ischemia induced by coronary artery ligation was used and the time to prepare the model lasted for 6 weeks. After completion of model preparation, administration of TSG was given for 6 weeks at a dose of 20 mg/kg. Test results demonstrate average artery pressure of ischemic animal in control group was lower than sham operation, while the blood pressure of animal in TSG administration group returned to the level of sham operation group. LVDP of ischemic control animal was higher than that of control group. But LVESP and ±dp/dtmax reduced. The corresponding hemadynamic indexes of TSG administration animal showed remarkable improvement. Additionally, the ischemic and infarct tissue of TSG administration animal reduced significantly in comparison with that in ischemic control animal. Test results indicate intragastric administration of TSG has significant therapeutic effect on chronic myocardial ischemia in rat.

Another embodiment of the present invention demonstrates LD50 of mouse tail intravenous injection of TSG is 648.94 mg/kg, of which 95% confidence limit is 571.18 mg/kg˜726.70 mg/kg.

Canine pharmacokinetics of single dose of TSG iv was observed in another embodiment of the present invention. The test results demonstrate after intravenous injection of TSG 10 mg/kg, 20 mg/kg, 30 mg/kg to healthy Beagle dogs, physiological disposition of TSG comply with two compartment model, of which the terminal elimination half lives (t1/2) of concentration-time curves were 168 min, 152 min, 373 min, respectively. AUC˜∞ were 315, 745 and 1552 μg·min/ml respectively, while AUC primarily has a positive correlation with dosage and the correlation coefficient r is 0.985.

The above study indicates TSG has obvious anti-myocardial ischemic effect, thus those skilled in the art can understand this compound has good applicable value for therapy and/or prevention of ischemic heart disease, i.e. CHD.

A series of tests of the present invention prove intravenous injection and/or oral administration of 3,4′,5-trihydroxy-stilbene-3-β-D-glucoside have remarkable therapeutic and/or preventive effects against myocardial ischemic injury and functional injury of heart induced by hypophysin and/or coronary artery ligation. The effects are characterized by reducing the extent and area of myocardial ischemic injury caused by ischemia, reducing the rise of serum LDH level caused by acute myocardial ischemia, recovering heart function and alleviating myocardial injury caused by chronic myocardial ischemia.

Different from reports on in vitro pharmacological test results, within the dosage range of the test examples of the current invention, TSG has no significant effect on heart rate and function of anesthetized normal animal. Thus in regard to living animal within such dosage range, TSG does not increase myocardial working, or cause noticeable change in myocardial consumption of oxygen resulted from positive chronotropic effect and positive inotropic effect. The research of the present invention suggests in dosage range of the test, TSG may increase coronary artery blood flow in test animal, and reduce the resistance and oxygen uptake rate of coronary artery. It is obvious such effects are helpful to anti-myocardial ischemia effect of TSG. There is neither report on such effects in the existing literature nor may such effects be inferred from the existing literature.

It is found in pharmacokinetic research in the maximum dose of 30 mg/kg used in the present invention, the maximum concentration in plasma obtained by TSG intravenous injection was only 100 μg/ml, around 0.25 mmol/L, differing from the concentration in plasma (5.25 mM) required for direct vasodilation in in vitro test by more than one magnitude order (Luo Sufang et al., Journal of First Military Medical University, 1992; 12(1); 10˜13). If a concentration of 5 mM in plasma is required, the dose of administration shall be 600 mg/kg. In fact, such dose is close to DL50 (640 mg/kg) of intravenous injection to mouse, so it is apparently impossible to practice in use. On the other hand, according to literature, TSG 0.02 mM˜2 mM may raise intracellular calcium concentration in vascular smooth muscle significantly. Therefore, the direct effect on vascular smooth muscle should be the positive inotropic effect and enhancement of angiotasis (Jin Chunhua et al., Chinese Pharmacological Bulletin 2000, 16(2); 151˜154). However, the whole animal test of the present invention proves TSG within the dosage range of the test has no significant effect on peripheral vascular resistance, which indicating TSG at the anti-myocardial ischemic doses of the present invention has no significant effect on in vivo resistance vessels.

It is further found in the present invention that continuous oral administration and intravenous injection of 3,4′,5-trihydroxy-stilbene-3-β-D-glucoside have the effect of anti-myocardial ischemia.

Therefore, the present invention raises: as anti-myocardial ischemic medicine, 3,4′,5-trihydroxy-stilbene-3-β-D-glucoside has beneficial value in use of preparation of medicine for therapy and/or prevention of coronary heart disease.

As there is no report relevent directly to the present invention in the existing literature and the existing basic research information does not indicate the potential value of TSG in anti-myocardial ischemia, and on the contrary, according to the test results of cultured myocardial cells and vascular smooth muscle cells in vitro (Jin Chunhua et al. Chinese Pharmacological Bulletin, 2000, 16(4): 400˜402; Jin Chunhua et al. Chinese Journal of Pathophysiology, 1998, 14(2): 195˜198; Jin Chunhua et al. Chinese Journal of Pathophysiology, 1999, 5(3): 233˜205; Jin Chunhua et al. Chinese Pharmacological Bulletin, 2000, 16(2): 151˜154), it is presumed TSG may not be conducive to the improvement of hemadynamics under the condition of myocardial ischemia and may aggravate the contradiction of blood supply and demand of ischemic myocardium. As the present invention proves there is no such positive inotropic effect and increase of angiotasis at the effective dosage of anti-myocardial ischemia as reported, it is obvious for technicians skilled in this art to understand the invention can be considered novelty and to involve an inventive step.

### BRIEF DESCRIPTION OF DRAWINGS

FIG. 1 displays the effect of TSG on the extent of canine myocardial ischemia (ΣST on epicardial electrogram) after coronary artery ligation:

Where: ordinates present the change rate of ΣST and abscissas indicate the time after coronary ligation.

Results: In comparison with solvent control group, the extent of myocardial ischemia in positive control group and all groups of TSG administration diminshes. (See Example 2 for details)

FIG. 2 displays the effect of TSG on the area of canine myocardial ischemia (N-ST on epicardial electrogram) after coronary artery ligation:

Where ordinates present the change rate of N-ST and abscissas indicate the time after coronary artery ligation.

Obviously, in comparison with solvent control group, the area of myocardial ischemia in positive control group and all groups of TSG administration reduces. (See Example 2 for details)

FIG. 3 displays the effect of TSG on myocardial oxygen uptake in anesthetized dogs.

Where ordinates present the change rate of myocardial oxygen uptake (%) and abscissas indicate the time after administration.

The figure proves that after administration, anesthetized dogs in all dose groups of TSG have their myocardial oxygen uptake decreased. (See Example 5 for details)

FIG. 4 displays the effect of TSG on coronary blood flow in anesthetized dogs.

Where ordinates present the change rate of coronary blood flow (%) and abscissas indicate the time after administration.

The figure proves that after administration, anesthetized dogs in all dose groups of TSG have their coronary blood flow increased (See Example 5 for details)

FIG. 5 displays the effect of TSG on cardiac output in anesthetized dogs.

Where ordinates present the change rate of cardiac output (%) and abscissas indicate the time after administration.

The figure proves that after middle and high dose groups of TSG administration, anesthetized dogs in these groups have their cardiac output increased (See Example 5 for details)

FIG. 6 displays the concentration-time curve after beagle dogs were medicated with a single dose of intravenous injection of TSG at the doses of 10, 20 and 30 mg/kg.

Where ordinates present the change rate of blood drug concentration (μg/ml) and abscissas indicate the time after administration. (See Example 9 for details)

### DETAILED DESCRIPTION OF THE INVENTION

The content of the present invention is further illustrated herein after in following examples. Unless explained particularly, the materials used in the examples were commercially purchased.

### Preparation of Test Samples of 3,4′,5-trihydroxy-stilbene-3-β-D-glucoside (TSG)

3,4′,5-trihydroxy-stilbene-3-β-D-glucoside (TSG) used in following examples was prepared with the method described in a Chinese patent application NEW METHOD FOR PREPARATION OF POLYDATIN AND RESVERATROL NO. 200310112538.3. (Batch no: 031011, prepared by Shenzhen Neptunus Pharmaceutical Co., Ltd.).

### (1) Preparation of Granules of 3,4′,5-trihydroxy-stilbene-3-β-D-glucoside (Samples Used in Example 1, 4 and 7)

Prescription:

| | |
|---|---|
| 3,4′,5-trihydroxy-stilbene-3-β-D-glucoside | 20 g |
| Starch | 40 g |
| Microcrystalline cellulose (PH301) | 40 g |
| Weight of granules | 100 g |

Preparation: 3,4′,5-trihydroxy-stilbene-3-β-D-glucoside is mixed with lactose and microcrystalline cellulose (PH301) till they form a homogeneous mixture. After addition of proper amount of achohole-water, the powder is made into granules before drying and harvest.

### (2) Preparation of solution of 3,4′,5-trihydroxy-stilbene-3-β-D-glucoside (Samples Used in Example 2, 3, 5, 6, 8 and 9)

| | |
|---|---|
| 3,4′,5-trihydroxy-stilbene-3-β-D-glucoside | 100 g |
| Dehydrated alcohol | 8 L |
| Solution of NaOH of pH 9 with water added to | 10 L |
| repacked in | 1000 ampouls or bottles |

Preparation: 40 g of 3,4′,5-trihydroxy-stilbene-3-β-D-glucoside is dissolved by heating in 8 L of dehydrated alcohol. Water solution of NaOH at pH 8.5 was added till the total volume reaches 10 L before being filtrated with a millipore filter and repacked into 1000 ampouls or bottles.

### EXAMPLE 1

### Effect of Oral Administration of 3,4′,5-trihydroxy-stilbene-3-β-D-glucoside on Myocardial Ischemia Induce by Hypophysin in Rats

The purpose of this example is to confirm the effect of oral administration of 3,4′,5-trihydroxy-stilbene-3-β-D-glucoside on myocardial ischemia induce by hypophysin in rats

### Drugs and Agents

Test drug: 3,4′,5-trihydroxy-stilbene-3-β-D-glucoside (batch no: 031011), prepared with 0.8% CMC into suspension at concentrations of 1.25, 2.5 and 5 mg/ml before administration.

Control drug: Dansheng Tablet (batch no: 030926), 300 mg/tab, product of Shanghai Lei Yun Shang Pharmaceutical Co., Ltd, prepared with 0.8% CMC into suspension at concentration of 20 mg/ml before administration.

### Test Animal

50 male SD rats with each weight 300˜425 g, provided by experimental animal center in Second Military Medical University.

### Groups and Administration

5 groups were set up for the test, including blank control group, Danshen Tablet group (300 mg/kg) and administration groups of 3,4′,5-trihydroxy-stilbene-3-β-D-glucoside at low (5 mg/kg), middle (10 mg/kg) or high (20 mg/kg) doses. Both test and control drugs were administered intragastrically in equal volume at unequal concentration. The volume for intragastric administration was 3 ml/kg for all groups. Doses of 3,4′,5-trihydroxy-stilbene-3-β-D-glucoside for low, middle and high dose groups were 5, 10 and 20 mg/kg, respectively. The dose for Danshen Tablet group was 300 mg/kg, while blank control group was given equal volume of 0.8% CMC.

### Test Method

50 healthy male SD rats were randomized into 5 groups and intragastric administration of drug and control solvents were performed once daily for 3 consecutive days. One hour after administration on the third day, animals were anesthetized with 3% pentobarbital sodium ip at the dose of 30 mg/kg. Normal electrocardiogram of V3 lead was recorded (with a ECG-6511 electrocardiograph, manufactured by Shanghai Photoelectrical Medical Electronic Apparatus Co., Ltd.) with labelling voltage set to 1 mV=1 cm, but the animal would not be used test if its basic ECG was abnormal. After stabilized for 15 min, the animal was quickly injected with hypophysin (batch no: 020601, produced by Shanghai Hefeng Pharmaceutical Co., Ltd, and prepared to 0.6 U/ml with normal saline before use) at the dose of 6 U/kg through lingual veins. Records of V3 lead ECG at the time points before injection of hypophysin, immediately after and 0.5, 1, 2, 5, 10, 15, 20, 30, 40, 50 and 60 min after the injection was made. ST segment elevation at all time points were taken for statistical treatment and changes of extent of ST segment elevation (ΔST, mV) and condition of death of animals were observed.

### Test Results

(1) General changes of ECG after intravenous injection of hypophysin: After injection of hypophysin, the main representation of ECG in rats was the quick elevation of ST segment, which reached the peak value only 15˜30 seconds later, and then began to drop gradually with T-wave being lowing or inversing, or ST segment being markedly suppressed, indicating the myocardial ischemia of endocardium and epicardium, or with frequent ventricular premature contraction, while high-degree or complete atrial ventricular block could also be observed.

(2) Effect of test drug on traumatic ST segment elevation: Changes (ΔST, mV) of the degree of ST segment elevation induced by injury from injection of hypophysin were taken as the indexes for judgement. The results demonstrated that 3,4′,5-trihydroxy-stilbene-3-β-D-glucoside at doses of 10 mg/kg and 20 mg/kg can significantly reduce ST segment elevation on ECG caused by myocardial injury from intravenous injection of hypophysin in anesthetized SD rats.

### Conclusion

3,4′,5-trihydroxy-stilbene-3-β-D-glucoside at doses of 10 mg/kg and 20 mg/kg can significantly reduce ST segment elevation on ECG caused by myocardial injury from intravenous injection of hypophysin in anesthetized SD rats, indicating that the chemical at the doses of 10 mg/kg and 20 mg/kg po is comparatively effective in prevention of myocardial ischemic injury caused by hypophysin.

TABLE 1
Effect of TSG on ST segment elevation (ΔST) induced by hypophysin in SD rats
( x̅ ± s, mV, n = 10)

| | | Danshen Tablet | TSG | TSG | TSG |
|---|---|---|---|---|---|
| | Solvent control | 80 mg/kg | 20 mg/kg | 10 mg/kg | 5 mg/kg |
| 30 s after | 0.096 ± 0.063 | 0.056 ± 0.047 | 0.110 ± 0.055 | 0.066 ± 0.22 | 0.075 ± 0.070 |
| administration | | | | | |
| 1 min after | 0.103 ± 0.071 | 0.078 ± 0.059 | 0.086 ± 0.062 | 0.081 ± 0.063 | 0.078 ± 0.072 |
| administration | | | | | |
| 2 min after | 0.093 ± 0.062 | 0.076 ± 0.061 | 0.075 ± 0.051 | 0.070 ± 0.055 | 0.083 ± 0.070 |
| administration | | | | | |
| 5 min after | 0.121 ± 0.071 | 0.080 ± 0.054 | 0.068 ± 0.060 | 0.069 ± 0.067 | 0.056 ± 0.048* |
| administration | | | | | |
| 10 min after | 0.142 ± 0.197 | 0.093 ± 0.061 | 0.073 ± 0.061 | 0.083 ± 0.070 | 0.076 ± 0.066 |
| administration | | | | | |
| 15 min after | 0.158 ± 0.074 | 0.086 ± 0.060* | 0.059 ± 0.040** | 0.066 ± 0.042** | 0.076 ± 0.058* |
| administration | | | | | |
| 20 min after | 0.135 ± 0.089 | 0.065 ± 0.032* | 0.049 ± 0.047* | 0.055 ± 0.046* | 0.072 ± 0.054* |
| administration | | | | | |
| 30 min after | 0.119 ± 0.068 | 0.079 ± 0.058 | 0.041 ± 0.036** | 0.074 ± 0.072 | 0.067 ± 0.079 |
| administration | | | | | |
| 40 min after | 0.137 ± 0.081 | 0.066 ± 0.057* | 0.052 ± 0.040** | 0.069 ± 0.068 | 0.102 ± 0.093 |
| administration | | | | | |
| 50 min after | 0.160 ± 0.090 | 0.078 ± 0.070* | 0.066 ± 0.058* | 0.079 ± 0.073* | 0.089 ± 0.081 |
| administration | | | | | |
| 60 min after | 0.174 ± 0.109 | 0.079 ± 0.092 | 0.054 ± 0.046** | 0.064 ± 0.056** | 0.088 ± 0.069* |
| administration | | | | | |

P < 0.05;
**P < 0.01 vs control group

### EXAMPLE 2

### Protective Action of 3,4′,5-trihydroxy-stilbene-3-β-D-glucoside on iv Against Ischemic Reperfusion Injury of Myocardium in Rats

The purpose of this example is to observe the effect of intravenous injection of 3,4′,5-trihydroxy-stilbene-3-β-D-glucoside on ischemic reperfusion injury of myocardium in SD rats.

### Test Drugs

Test drug: 3,4′,5-trihydroxy-stilbene-3-β-D-glucoside (batch no: 031030302), diluted to 100 mg/10 ml with normal saline before use.

Positive control drug: sorbide nitrate injection (Isosorbide Dinitrate, batch no: 479210) manufactured by Germany Schwarz Pharma AG and repacked by Zhuhai Schwarz Pharma Co., Ltd.

### Test Animal

Male SD rats of SPF grade with each weight 250˜300 g, provided by experimental animal center in First Military Medical University.

### Groups and Administration

Sham operation group, normal saline control group and Isosorbide Dinitrate control group (0.6 mg/kg) and 3,4′,5-trihydroxy-stilbene-3-β-D-glucoside groups of low (7.5 mg/kg), middle (15 mg/kg) and high (30 mg/kg) doses (calculated with method of surface area, by which low dose of 7.5 mg/kg was equal to a dose of 100 mg for a human being weighing 70 kg) were set up for the test. Administration was performed by femoral intravenous injection.

### Test Method

(1) Method of operation and ligation of coronary artery: Male SD rats were anesthetized with pentobarbital sodium (45 mg/kg) and immobilized on their backs for records of II lead ECG with an electrocardiograph (ECG-6851C, manufactured by Shanghai Photoelectrical Medical Electronic Apparatus Co., Ltd.). Tracheas were intubated and connected to a breathing apparatus. Thoracotomy was performed at intercostal space on left chest for cutting down fourth/fifth rib and opening cardiac pericardium to expose the heart. A 0/3 suture line was passed underneath the left branch of anterior descending coronary artery. After 10 minutes of stabilization (those with abnormal ECG after stabilization were abandoned). With two thrums passing through a small piece of fine silicone tube, a ligature was made on another small piece of fine silicone tube for ischemic ligation (those with no changes in ST segment and T-wave were excluded). After 10 minutes of ligation, drugs were injected slowly through femoral veins. 40 minutes later, the lines for ligation were cut off to reperfuse the anterior descending branch for 30 minutes.

(2) Detection of lactate dehydrogenase (LDH) and creatine kinase (CK): After completion of reperfusion, blood was sampled from abdominal aorta to determine serum activity of LDH and CK with ultraviolet spectrometry.

(3) Measurement of the area of myocardial infarction: With same thickness, 5 slices of ventricular myocardium were cut off from the part under the line of coronary ligation and in parallel to atrioventricular furrow and put into staining solution of nitroblue tetrazolium (N-BT) for 15 min of shaking dyeing. Normal myocardium would be dyed to lasureous color, while the infarct part of myocardium would not be dyed and remain pale-yellow. Under a microscopy, the infarct part was separated and weighed, and the percentage (%) of the weight of infarct part to the weight of total myocardium was adopted as the index for area of myocardial infarction.

(4) Data and statistic treatment: Data in all groups were presented with x̅±s and analysis of variance was adopted to check their significance.

### Test Results

After myocardial ischemic reperfusion, serum LDH and CK activities and the weight of infarct area were all increased obviously, and the difference was of great significance in comparison with sham operation group (P<0.01). In low, middle and high dose groups (7.5, 15, 30 mg/kg) of 3,4′,5-trihydroxy-stilbene-3-β-D-glucoside, the serum LDH and CK activities and the weight of infarct area of myocardium were obviously lower than those in model groups (P<0.05). In middle and high dose groups (15 mg/kg, 30 mg/kg) of 3,4′,5-trihydroxy-stilbene-3-β-D-glucoside, the serum LDH activities were obviously lower than those in model groups (P<0.05). Similar to groups of 3,4′,5-trihydroxy-stilbene-3-β-D-glucoside, serum LDH and CK activities and the weight of infarct area were obviously reduced in Isosorbide Dinitrate group (P<0.05). (See Table 2)

### Conclusion

Intravenous injection of 3,4′,5-trihydroxy-stilbene-3-β-D-glucoside has protective action against myocardial injury caused by ischemic reperfusion in rats. It can inhibit the efflux of LDH and CK when myocardium injury occurs during the time of ischemic reperfusion, and reduce the activities of LDH and CK in serum and the weight of infarct area of myocardium, while presenting some dose-effect relationship.

TABLE 2
Effect of TSG on serum activities and extent of myocardial infarction in ischemic
reperfusion rats ( x̅ ± s, n = 10)

| | | | | Infarct area |
|---|---|---|---|---|
| Groups | Doses | CK (IU/L) | LDH (IU/L) | percentage (%) |
| Sham operation | 1.0 ml/kg | 510.8 ± 80.9 | 655.5 ± 78.3 | — |
| Normal saline | 1.0 ml/kg | 865.4 ± 189.1^{ΔΔΔ} | 989.9 ± 184.6^{ΔΔΔ} | 33.73 ± 3.83^{ΔΔΔ} |
| Isosorbide | 0.6 mg/kg | 637.1 ± 100.4*** | 806.0 ± 92.0** | 23.98 ± 3.16*** |
| Dinitrate | | | | |

3,4′,5-trihydroxy-stilbene-3-β-D-glucoside groups

| | | | | |
|---|---|---|---|---|
| Low dose | 7.5 mg/kg | 710.7 ± 106.8** | 857.7 ± 124.0 | 27.57 ± 5.33*** |
| Middle dose | 15.0 mg/kg | 630.6 ± 108.6*** | 809.4 ± 128.4** | 24.80 ± 3.32*** |
| High dose | 30.0 mg/kg | 559.4 ± 72.9*** | 744.0 ± 149.2*** | 23.71 ± 3.66*** |

**P < 0.05,
***P < 0.01 in comparison with normal saline group;
^{ΔΔΔ}P < 0.01 in comparison with sham operation group

### EXAMPLE 3

### Therapeutic Effect of 3,4′,5-trihydroxy-stilbene-3-β-D-glucoside iv on Canine Model of Myocardial Infarction Induced by Ligation of Coronary Artery

The purpose of the present example is to observe therapeutic effect of 3,4′,5-trihydroxy-stilbene-3-β-D-glucoside on canine model of myocardial infarction induced by ligation of coronary artery.

### Test Drugs

Test drug: 3,4′,5-trihydroxy-stilbene-3-β-D-glucoside (Batch no: 03030302), 100 mg/10 ml; diluted with normal saline before use.

Positive control drug: sorbide nitrate injection (Isosorbide Dinitrate, batch no: 479210) manufactured by Germany Schwarz Pharma AG and repacked by Zhuhai Schwarz Pharma Co., Ltd.

### Test Animal

Healthy male and female hybrid dogs with each weight 10˜15 kg, provided by experimental animal center in First Military Medical University.

### Groups and Administration

Sham operation group, normal saline control group and Isosorbide Dinitrate control group (0.4 mg/kg) and groups for low (2.5 mg/kg), middle (5 mg/kg) and high (10 mg/kg) doses of 3,4′,5-trihydroxy-stilbene-3-β-D-glucoside were set up for the test. Drugs were medicated with intravenous injection. The volume of the drug administered was the same as that of normal saline for the animal model group of ischemic reperfusion with administrative volume of 2 ml/kg.

### Test Method

After weighing, the animals were anesthetised with pentobarbital sodium iv at the dose of 30 mg/kg. Tracheal intubation was performed and SC-M5 anesthesia respirator (manufactured by Shanghai Medical Instrument Factory) was connected for mechanical ventilation (16˜18 per min with tidal volume of 350˜550 ml) after thoracotomy at the fourth intercostal space of left sternal border. With exposure of the heart, the pericardium was cut open to make pericardium hammock. The coronary artery was dissociated from between the first and second branches of left anterior descending coronary artery, underneath which passed through silk threads for two-step ligation. On surface of heart were placed 30-point epicardial leads, to which four limbs were connected through needle-electrodes, which in turn were connected to Powerlab/8s (AD Instruments) with a multichannel switcher. Harris Two-step Ligation was performed. Two minutes before first ligation, 5 mg/kg lidocaine was given by femoral intravenous injection to prevent arrhythmia. While performing ligation, a steel wire of 1 mm in diameter was inserted into the first knot so as to be ligated together with the coronary artery. Afterward, the steel-wire was pulled out and then 30 min later, the second knot was made to complete the ligation.

The epicardial electrogram 10 min after completing the ligation was recorded as the control value before drug administration. Later, test drug was given by femoral intravenous injection while the negative control group was given solvent of same volume. Constant dripping was completed within 30 min for all groups with a constant flow pump (SH-88AB Controllable Intravenous Injector made by Quanzhou Lizhong Electronic Medical Instrument Factory). After completion of the constant dripping, changes of epcardial electrogram at 5, 15, 30, 60, 90 and 120 min after administration were recorded respectively. The number of ST segment elevation or depression to more than 2 mV and sum of total value ST segment elevation (ΣST) were applied as the indexes for observation of changes of epcardial electrogram and to calculate the percentage of the weight of infarct area to the weight of whole heart or to the weight of left ventricle. 3 ml of blood from right ventricle was sampled before ligation and 2 hrs after administration. After centrifuged at 3000 rpm for 15 min, serum was taken to detect lactate dehydrogenase (LDH) and serum creatine kinase (CK). Method according to instruction of the LDH and CK Assay kit (batch no: 20020523, Nanjing Jiancheng Bioengineering Institute) (ENR: U90625, Randox Company, UK) was adopted for detection. The performance was with a ultraviolet/visible range spectrophotometer of UV751GD type (Shanghai analytical apparatus Factory).

### Test Results

(1) Effects of 3,4′,5-trihydroxy-stilbene-3-β-D-glucoside (TSG) on extent of myocardial ischemia (ΣST in epcardial electrogram): 5 min after coronary artery ligation, the sum of ST segment (ΣST) of epcardial electrogram in solvent control group was obviously elevated. At the time of 30 min after ligation, ΣST raised high up to 18.2% in solvent control group and then dropped slowly afterward. Intravenous administration of TSG at the dose of 2.5˜10 mg/kg can dose-dependently relieve the extent of myocardial ischemia and decrease ΣST obviously and there was significant difference between low dose group and solvent control group at the time points of 30˜90 min. Middle dose group had also significant difference at all time points in comparison with solvent control group. As for high dose group, obvious effect was produced immediately after administration and lasted to 120 min. Isosorbide Dinitrate group had ΣST dropped 5 to 90 min after administration, but there was no significant difference at the time points from 90 min to 120 min in comparison with solvent control group. The results of experiment demonstrate that intravenous administration of TSG at 2.5, 5 and 10 mg/kg has obvious therapeutic effects on extent of canine acute myocardial ischemia induced by coronary artery ligation. (See Table 3, FIG. 1)

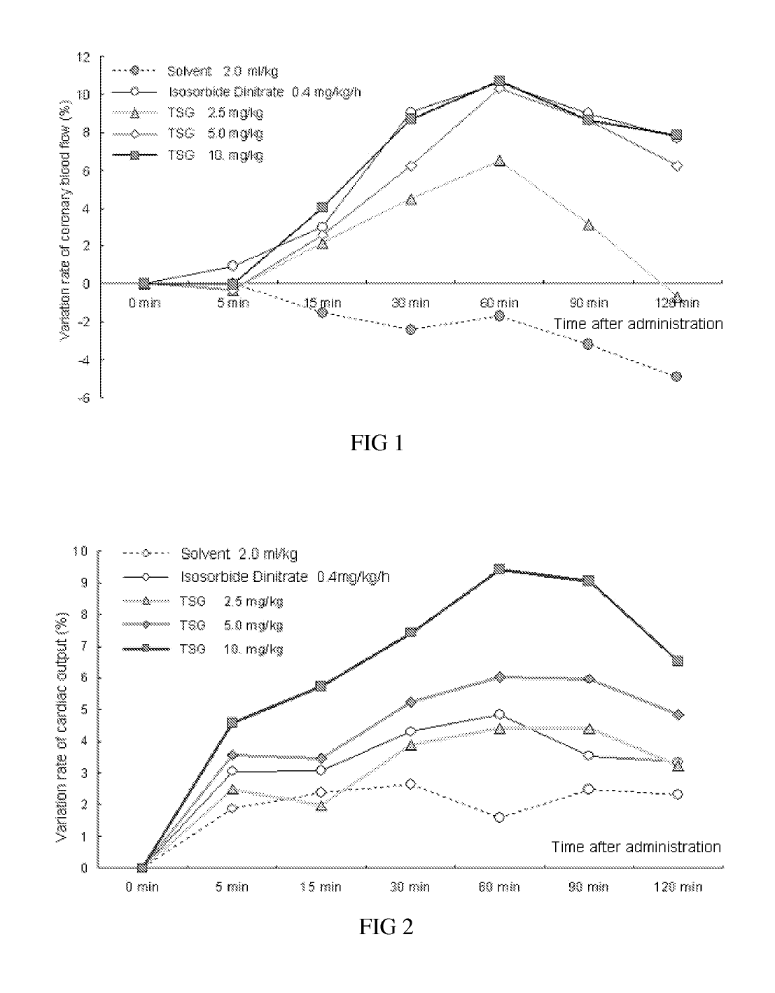

(2) Effects of 3,4′,5-trihydroxy-stilbene-3-β-D-glucoside (TSG) on area of myocardial ischemia (N-ST in epcardial electrogram): TSG can dose-dependently reduce the area of myocardial ischemia and the value of N-ST and the time of such effect can last to 120 min. In comparison of the rates of N-ST reduction at all time points in low dose group of 2.5 mg/kg to those in solvent control group at the same time points, there was significant difference from 30˜90 min (P<0.05). There was also significant difference from 15˜120 min in the rates of N-ST reduction in middle dose group of 5 mg/kg at all time points when compared with those of solvent control group at the same time points. In high dose group (10 mg/kg), there was very significant difference in reduction of myocardial ischemia within the time of 5˜120 min in comparison with solvent control group. The area of myocardial ischemia in Isosorbide Dinitrate group dropped remarkably 5 min after administration in comparison with solvent control group at the same time points. (See Table 4, FIG. 2)

(3) Effects of 3,4′,5-trihydroxy-stilbene-3-β-D-glucoside (TSG) on quantitative histological detection of the area of myocardial infarction: The quantitative histological detection of the area of myocardial infarction is to adopt N-BT staining to demonstrate the effect of 3,4′,5-trihydroxy-stilbene-3-β-D-glucoside on the area of myocardial infarction, the results of which is the same as those detected with epcardial electrogram. Intravenous administration of TSG at the dose of 2.5˜10 mg/kg can dose dependently reduce the area of myocardial infarction. The ratios of infarct zone/left ventricle (IZ/LV) in low, middle and high dose (2.5, 5, 10 mg/kg) groups reduced significantly in comparison with those of solvent control group (P<0.01). Compared with solvent control group, the ratios of infarct zone/whole heart (IZ/WH) decreased significantly in low dose group (P<0.05) and very significantly in middle and high dose groups (P<0.01). The effect in high dose group was the strongest. Isosorbide Dinitrate group had also their areas of myocardial infarction reduced significantly (P<0.01). (See Table 5)

(4) Effects of 3,4′,5-trihydroxy-stilbene-3-β-D-glucoside (TSG) on serum biochemical indicators after coronary artery ligation: After coronary artery ligation, both serum lactate dehydrogenase (LDH) and creatine kinase (CK) rose in all groups. TSG can reduce the degree of serum LDH increment very significantly in low, middle and high dose groups and in solvent control group (P<0.01), and it can reduce the degree of serum CK increment very significantly in middle and high dose groups and in solvent control group (P<0.01), while reducing the degree of serum CK increment significantly in low dose group (P<0.05). Among all groups, the high dose (10 mg/kg) group demonstrated the strongest pharmacodynamic action. (See Table 6)

### Conclusion

The experimental results proved that intravenous injection of 3,4′,5-trihydroxy-stilbene-3-β-D-glucoside (TSG) can obviously reduce the extent of canine myocardial ischemia induced by coronary artery ligation and minimize the area of myocardial ischemia. The intensity of such action is dose-dependent. The results of quantitative detection of histology are the same as those detected with epicardial electrogram. In comparison with solvent control group, infarct zone minimizes remarkably, demonstrating that TSG intravenous injection has obvious therapeutic effects on canine myocardial ischemia induced by coronary artery ligation. The results of experiment also prove that 3,4′,5-trihydroxy-stilbene-3-β-D-glucoside can significantly reduce the degrees of serum increment of lactate dehydrogenase and creatine kinase, indicating that it can diminish the leakage of LDH and CK from cadiocyte, and minimize the cellular damage resulted from myocardial ischemia and has protective effect on myocardial cells.

TABLE 3
Effect of TSG on sum of ST (ΣST) segment elevation in epicardial electrogram of dogs
with coronary artery ligation (mV, x̅ ± s, n = 6)

| | | Before | | | | | | |
|---|---|---|---|---|---|---|---|---|
| | | administration | After administration | | | | | |
| Groups | Indexes | 0 min | 5 min | 15 min | 30 min | 60 min | 90 min | 120 min |
| Solvent control | x̅ ± s | 153.3 ± 43.7 | 175.7 ± 51.7 | 177.8 ± 40.7 | 178.8 ± 44.0 | 173.0 ± 42.1 | 165.5 ± 41.7 | 148.5 ± 43.2 |
| group | variation % | | 14.7 ± 7.4 | 17.7 ± 10.1 | 18.2 ± 10.6 | 14.5 ± 11.7 | 8.8 ± 3.9 | −3.1 ± 7.8 |
| 2 ml/kg | | | | | | | | |
| Isosorbide | x̅ ± s | 160.5 ± 41.2 | 149.3 ± 36.7 | 128.7 ± 46.6 | 111.2 ± 44.5 | 111.0 ± 38.2 | 115.8 ± 29.7 | 138.5 ± 45.5 |
| Dinitrate | variation % | | −6.7 ± 4.3** | −20.9 ± 13.8** | −32.6 ± 11.0** | −31.4 ± 7.6** | −27.4 ± 6.7** | −13.7 ± 18.0 |
| 0.4 mg/kg/h | | | | | | | | |
| TSG | x̅ ± s | 154.5 ± 60.4 | 154.7 ± 55.0 | 149.5 ± 52.8 | 129.2 ± 57.7 | 122.7 ± 59.6 | 128.3 ± 55.1 | 130.2 ± 54.1 |
| 2.5 mg/kg | variation % | | 1.5 ± 9.5 | −1.1 ± 20.3 | −17.4 ± 8.5** | −22.2 ± 12.3** | −17.3 ± 12.4* | −15.5 ± 16.8 |
| TSG | x̅ ± s | 153.8 ± 50.7 | 148.0 ± 39.9 | 133.7 ± 52.7 | 116.2 ± 53.7 | 105.3 ± 53.6 | 103.8 ± 54.1 | 100.5 ± 48.3 |
| 5.0 mg/kg | variation % | | −1.7 ± 10.3** | −11.7 ± 23.2* | −25.4 ± 17.8** | −32.6 ± 19.5** | −33.2 ± 21.9** | −32.1 ± 24.8* |
| TSG | x̅ ± s | 155.0 ± 46.4 | 151.7 ± 35.6 | 132.0 ± 43.1 | 115.2 ± 50.9 | 104.7 ± 45.5 | 102.0 ± 51.8 | 103.3 ± 46.4 |
| 10 mg/kg | variation % | | −0.7 ± 7.7* | −15.4 ± 5.6* | −28.0 ± 12.8** | −34.1 ± 13.0** | −37.1 ± 15.7** | −35.3 ± 14.3* |

*P < 0.05,
**P < 0.01 in comparison with solvent control group

TABLE 4
Effect of TSG on canine myocardial infarction area (N-ST) induced by coronary artery ligation ( x̅ ± s, n = 6)

| | | Before | | | | | | |
|---|---|---|---|---|---|---|---|---|
| | | administration | After administration | | | | | |
| Groups | Indexes | 0 min | 5 min | 15 min | 30 min | 60 min | 90 min | 120 min |
| Solvent control | x̅ ± s | 26.5 ± 4.5 | 28.2 ± 3.9 | 28.3 ± 2.7 | 29.2 ± 2.9 | 29.5 ± 1.2 | 28.3 ± 1.6 | 27.0 ± 2.2 |
| group 2 ml/kg | variation % | | 6.5 ± 5.4 | 7.2 ± 6.0 | 10.5 ± 9.4 | 11.9 ± 9.1 | 9.4 ± 10.6 | 2.3 ± 9.8 |
| Isosorbide | x̅ ± s | 28.2 ± 3.2 | 26.2 ± 2.7 | 23.2 ± 6.7 | 20.0 ± 5.9** | 21.5 ± 1.5** | 22.2 ± 2.4** | 22.8 ± 2.4 |
| Dinitrate | variation % | | −7.0 ± 8.3* | −17.9 ± 10.7** | −28.9 ± 14.3** | −23.6 ± 3.9** | −21.3 ± 7.1** | −18.8 ± 8.5* |
| 0.4 mg/kg/h | | | | | | | | |
| TSG | x̅ ± s | 26.7 ± 4.1 | 27.5 ± 4.5 | 26.0 ± 5.6 | 23.2 ± 5.3* | 21.8 ± 3.8** | 23.8 ± 2.3 | 23.8 ± 3.9 |
| 2.5 mg/kg | variation % | | 3.4 ± 11.7 | −2.5 ± 10.0 | −12.6 ± 11.1* | −18.4 ± 10.3** | −10.7 ± 3.0* | −10.8 ± 11.1 |
| TSG | x̅ ± s | 26.7 ± 3.9 | 25.8 ± 3.7 | 24.3 ± 5.1 | 21.0 ± 8.2** | 19.0 ± 4.2** | 19.7 ± 5.2** | 17.8 ± 4.0** |
| 5.0 mg/kg | variation % | | −3.0 ± 7.4 | −8.3 ± 14.3* | −20.6 ± 17.9** | −28.3 ± 16.7** | −25.4 ± 21.2** | −32.9 ± 15.5** |
| TSG | x̅ ± s | 27.8 ± 3.1 | 24.8 ± 6.4 | 22.2 ± 8.4 | 18.7 ± 6.8** | 18.7 ± 2.5** | 19.2 ± 1.9** | 20.2 ± 1.8** |
| 10 mg/kg | variation % | | −10.9 ± 12.7** | −20.5 ± 12.7** | −32.9 ± 18.6** | −32.8 ± 9.1** | −31.0 ± 8.0** | −27.6 ± 4.9** |

*P < 0.05,
**P < 0.01 in comparison with solvent control group

TABLE 5
Effect of TSG on area of myocardial infarction detected with
N-BT staining ( x̅ ± s, n = 6)

| Groups | IZ/WT(%) | IZ/LV (%) |
|---|---|---|
| Solvent control group 2 ml/kg | 14.84 ± 2.18 | 26.15 ± 1.55 |
| Isosorbide Dinitrate 0.4 mg/kg/h | 9.57 ± 2.48* | 15.47 ± 3.80** |
| TSG 2.5 mg/kg | 11.70 ± 0.93* | 21.68 ± 1.88** |
| TSG 5.0 mg/kg | 9.03 ± 1.16** | 15.25 ± 1.41** |
| TSG 10 mg/kg | 8.65 ± 1.11** | 13.92 ± 1.32** |

*P < 0.05,
**P < 0.01 in comparison with solvent control group

TABLE 6
Effect of TSG on serum biochemical indicators after coronary artery ligation ( x̅ ± s, n = 6)

| | | LDH(IU/L) | | | CK(IU/L) | | |
|---|---|---|---|---|---|---|---|
| | | Before | After | | Before | After | |
| Groups | | administration | administration | B/A | administration | administration | B/A |
| Solvent control group | 2 ml/kg | 326.8 ± 55.1 | 919.6 ± 211.2 | 2.81 ± 0.35 | 171.3 ± 25.8 | 526.2 ± 102.9 | 3.07 ± 0.37 |
| Isosorbide Dinitrate | 0.4 mg/kg/h | 330.0 ± 78.4 | 663.1 ± 133.3 | 2.03 ± 0.22** | 172.7 ± 13.2 | 360.2 ± 51.9 | 2.09 ± 0.25** |
| TSG | 2.5 mg/kg | 359.7 ± 70.5 | 823.3 ± 186.3 | 2.28 ± 0.21** | 176.7 ± 18.8 | 446.6 ± 39.4 | 2.54 ± 0.27* |
| TSG | 5.0 mg/kg | 281.2 ± 80.9 | 587.1 ± 170.6 | 2.09 ± 0.16** | 180.8 ± 13.2 | 356.2 ± 64.8 | 1.98 ± 0.38** |
| TSG | 10 mg/kg | 279.3 ± 70.8 | 551.6 ± 131.3 | 1.99 ± 0.14** | 172.7 ± 11.1 | 299.5 ± 63.9 | 1.74 ± 0.37** |

*P < 0.05,
**P < 0.01 in comparison with solvent control group

### EXAMPLE 4

### Protective Action of Oral Administration of 3,4′,5-trihydroxy-stilbene-3-β-D-glucoside Against Myocardial Infarction in Anesthetized Dogs

The purpose of the present example is to observe the protective action of intragastric administration of 3,4′,5-trihydroxy-stilbene-3-β-D-glucoside against acute myocardial infarction in dogs.

### Test Drugs

Test drug: 3,4′,5-trihydroxy-stilbene-3-β-D-glucoside (batch no: 031019), prepared with 0.8% CMC into suspension with concentration of 1, 2, 4 mg/ml before administration.

Control drug: Danshen Tablet (batch no: 030926), 300 mg/tab, product of Shanghai Lei Yun Shang Pharmaceutical Co., Ltd.

### Test Animal

30 healthy male and female hybrid dogs with each weight 12˜14 kg, provided by experimental animal center in First Military Medical University.

### Groups and Administration

Solvent control group, positive control group (Dansheng Tablet 45 mg/kg) and groups of 3,4′,5-trihydroxy-stilbene-3-β-D-glucoside at doses of 5, 10 and 20 mg/kg. Drugs were administered intragastrically in equal volume at unequal concentration. The volume for intragastric administration was 5 ml/kg and the drugs for all aforesaid groups were given intragastrically 30 min after indexes got stabilized in records.

### Test Method

After weighing, animals were anesthetized with 3% pentobarbital sodium iv at the dose of 30 mg/kg. Tracheal intubation was performed and anesthesia respirator (SC-M5, manufactured by Shanghai Medical Instrument Factory) was connected for mechanical ventilation (18˜20 per min with tidal volume of 350˜550 ml) after thoracotomy. Needle electrodes were punctured into four limbs and under chest skin for monitoring standard limb lead and V3 and ECG. Thoracotomy was performed at the third intercostal space of left sternal border and the fourth rib was removed for full exposure of heart. The pericardium was cut open to make a pericardium hammock. The coronary artery was dissociated from between the second and third branches of left anterior descending coronary artery, underneath which pass through silk threads for two-step ligation. On surface of heart were placed 30-point epicardial leads, to which four limbs were connected through needle-electrodes, which were in turn connected to Powerlab/8s (AD Instruments) with a multichannel switcher to record epicardial electrogram.

Animal models of acute myocardial ischemia were prepared with the method of two-step ligation. Two minutes before first ligation, 5 mg/kg lidocaine was given by femoral intravenous injection to prevent arrhythmia. 5, 15, 30, 60, 90, 120, 180 min after administration of normal saline or test drug, epicardial electrogram was recorded at 30 mapping points and ST segment elevation exceeding 2 mV were taken as the criteria to judge and calculate the extent of myocardial ischemia (Σ-ST total millivolts of ST segment elevation) and the area of myocardial ischemia (N-ST total points of ST segment elevation exceeding 2 mV). 180 min later, 4 ml of blood was drawn from femoral vein and centrifuged for 5 minutes at 10000 rpm before serum was taken and stored at −18° C. for detection of LDH (LDH kit, batch no: 20020523, Nanjing Jiancheng Bioengineering Institute). The heart was taken and weight before auricle and right ventricle were cut off to weight the left heart, which was then sliced to pieces of 0.5˜1 cm in thickness. After being cleaned with normal saline, the myocardial slices were stained at 37□ with Nitro-tetrazolium Blue chloride, N-BT, produced by Swiss Fluka Chemicals and Agents Co., Ltd. NBT). While other part of myocardium was dyed to blue-black, the Infarct myocardium would not be colorated, which was then cut off and weighted.

The results of experiment were shown in x̅±s and statistics were checked with t-test of non-matched pair. When p<0.05, it was judged that the differences were statistically significant.

### Test Results

(1) Effect of TSG on heart rate (surface electrocardiogram) of anesthetized dogs: there were no obvious changes in solvent control group, positive control group and the groups of TSG at three doses before and after administration (See Table 7).

TABLE 7
Effect of oral TSG on heart rates of anesthetized dogs ( x̅ ± s, time/min, n = 6)

| | Solvent control | Danshen Tablet group | TSG | TSG | TSG |
|---|---|---|---|---|---|
| | group | 45 mg/kg | 5 mg/kg | 10 mg/kg | 20 mg/kg |
| Before | 164 ± 15 | 170 ± 12 | 173 ± 16 | 169 ± 12 | 185 ± 10 |
| administration | | | | | |
| 5 min after | 161 ± 12 | 176 ± 13 | 174 ± 18 | 169 ± 11 | 186 ± 13 |
| administration | | | | | |
| 15 min after | 167 ± 17 | 175 ± 11 | 168 ± 17 | 167 ± 10 | 182 ± 11 |
| administration | | | | | |
| 30 min after | 164 ± 18 | 173 ± 14 | 170 ± 16 | 165 ± 12 | 184 ± 12 |
| administration | | | | | |
| 60 min | 168 ± 16 | 167 ± 13 | 167 ± 12 | 163 ± 16 | 178 ± 16 |
| 90 min after | 165 ± 11 | 168 ± 16 | 165 ± 12 | 165 ± 12 | 177 ± 15 |
| administration | | | | | |
| 120 min after | 166 ± 18 | 172 ± 13 | 167 ± 13 | 162 ± 11 | 180 ± 21 |
| administration | | | | | |
| 180 min after | 169 ± 18 | 164 ± 16 | 163 ± 12 | 159 ± 14 | 168 ± 19 |
| administration | | | | | |

(2) Effect of TSG on extent of ST segment elevation (ΔST) in Surface ECG of myocardial ischemia dogs: Intragastric administration of Dansheng tablet and TSG at all doses can reduce ST segment elevation induced by myocardial ischemia. TSG group of 20 mg/kg achieved significant result (See Table 8).

(3) Effect of TSG on extent of acute myocardial ischemia (Σ-ST in epicardial electrogram) in anesthetized dogs: In comparison with solvent control group, Σ-ST in all dose groups dropped to different degrees. In most time periods after intragastric administration, ST segment dropped significantly (See Table 9).

(4) Effect of TSG on area of acute myocardial ischemia (N-ST in epicardial electrogram) of anesthetized dogs: showed As percentage of ST segment exceeding 2 mV in 32 electrodes, changes of N-ST were not significant in solvent control group after administration and it dropped gradually in Danshen Tablet group and TSG groups at all doses after administration. In comparison with solvent control group at the same time period, the reductions were significant 90 minutes after administration (See Table 10).

TABLE 8
Effect of oral TSG on ΔST of dogs ( x̅ ± s, mV, n = 6)

| | Solvent control | Dansheng tablet group | TSG | TSG | TSG |
|---|---|---|---|---|---|
| | group | 45 mg/kg | 5 mg/kg | 10 mg/kg | 20 mg/kg |
| Before administration | 1.10 ± 0.60 | 0.41 ± 0.34* | 0.66 ± 0.45 | 0.55 ± 0.40 | 0.58 ± 0.30 |
| 5 min after administration | 1.04 ± 0.63 | 0.77 ± 0.42 | 0.70 ± 0.50 | 0.68 ± 0.51 | 0.45 ± 0.17* |
| 15 min after administration | 1.21 ± 0.80 | 0.60 ± 0.30 | 0.71 ± 0.48 | 0.69 ± 0.52 | 0.39 ± 0.16* |
| 30 min after administration | 1.09 ± 0.48 | 0.71 ± 0.43 | 0.73 ± 0.51 | 0.69 ± 0.45 | 0.35 ± 0.19* |
| 60 min | 1.30 ± 0.75 | 0.69 ± 0.44 | 0.88 ± 0.54 | 0.70 ± 0.44 | 0.26 ± 0.15** |
| 90 min after administration | 1.32 ± 0.68 | 0.64 ± 0.38 | 0.82 ± 0.49 | 0.75 ± 0.46 | 0.28 ± 0.13** |
| 120 min after administration | 1.40 ± 0.71 | 0.55 ± 0.33* | 0.86 ± 0.52 | 0.81 ± 0.52 | 0.23 ± 0.16** |

*p < 0.05,
**P < 0.01 vs solvent control group.

TABLE 9
Effect of oral TSG on extent of myocardial ischemia (Σ-ST) in dogs
( x̅ ± s, mV, n = 6)

| | Solvent control | Dansheng tablet group | TSG | TSG | TSG |
|---|---|---|---|---|---|
| | group | 45 mg/kg | 5 mg/kg | 10 mg/kg | 20 mg/kg |
| Before administration | 55.6 ± 9.11 | 59.2 ± 7.03 | 55.7 ± 6.14 | 54.3 ± 1.89 | 62.1 ± 6.34 |
| 5 min after administration | 92.1 ± 12.8 | 82.0 ± 13.7 | 77.3 ± 15.0 | 77.9 ± 10.6 | 78.7 ± 5.68* |
| 15 min after administration | 95.0 ± 8.23 | 77.9 ± 14.7* | 77.9 ± 13.9* | 69.6 ± 7.90*** | 77.8 ± 8.49** |
| 30 min after administration | 93.0 ± 6.81 | 72.2 ± 12.7** | 71.6 ± 8.71*** | 69.8 ± 7.32*** | 70.6 ± 4.79*** |
| 60 min after administration | 87.3 ± 5.59 | 70.9 ± 10.9** | 72.2 ± 8.68** | 75.9 ± 16.3 | 68.7 ± 6.25*** |
| 90 min after administration | 90.6 ± 11.4 | 69.1 ± 12.4* | 70.5 ± 8.46** | 68.8 ± 7.83** | 65.7 ± 7.12** |
| 120 min after administration | 89.1 ± 5.58 | 66.0 ± 7.68*** | 73.6 ± 8.00** | 66.3 ± 5.60*** | 65.0 ± 6.12*** |
| 180 min after administration | 82.4 ± 8.82 | 61.7 ± 5.17*** | 67.4 ± 8.19* | 64.2 ± 5.45** | 62.5 ± 3.03*** |

*P < 0.05,
**P < 0.01,
***P < 0.001 vs solvent control group

TABLE 10
Effect of oral TSG on canine N-ST ( x̅ ± s, %, n = 6)

| | Solvent control | Danshen Tablet group | TSG | TSG | TSG |
|---|---|---|---|---|---|
| | group | 45 mg/kg | 5 mg/kg | 10 mg/kg | 20 mg/kg |
| Before administration | 34.6 ± 13.7 | 35.2 ± 19.3 | 26.5 ± 17.6 | 22.8 ± 9.44 | 42.3 ± 20.6 |
| 5 min after administration | 85.6 ± 19.3 | 86.4 ± 13.69 | 70.3 ± 21.4 | 79.5 ± 19.5 | 89.2 ± 20.3 |
| 15 min after administration | 79.6 ± 14.3 | 81.1 ± 16.3 | 66.9 ± 19.8 | 72.3 ± 15.9 | 78.9 ± 13.7 |
| 30 min after administration | 81.0 ± 15.5 | 76.1 ± 16.2 | 70.3 ± 16.3 | 69.4 ± 13.2 | 74.9 ± 12.9 |
| 60 min after administration | 85.2 ± 8.03 | 66.3 ± 19.8 | 69.5 ± 15.8 | 68.0 ± 18.8 | 67.2 ± 16.5 |
| 90 min after administration | 86.3 ± 9.89 | 68.9 ± 16.6* | 63.0 ± 18.7* | 68.6 ± 15.6* | 59.2 ± 18.9* |
| 120 min after administration | 83.4 ± 8.69 | 57.9 ± 16.8* | 63.2 ± 12.8* | 61.9 ± 14.9* | 56.4 ± 13.6** |
| 180 min after administration | 80.1 ± 17.2 | 49.6 ± 18.6** | 58.3 ± 19.3 | 55.5 ± 16.7* | 46.8 ± 14.9** |

N-ST: percentage of ST segment exceeding 2 mV in 32 electrodes;
*P < 0.05,
**P < 0.01 vs solvent control group.

(5) Effect of TSG on serum enzymology after acute myocardial infarction in anesthetized dogs: In comparison with solvent control group, TSG at the doses of 10 and 20 mg/kg can reduce the level of LDH remarkably after intragastric administration (See Table 11).

TABLE 11
Effect of oral TSG on LDH after acute myocardial infarction in
anesthetized dogs ( x̅ ± s, IU/L, n = 6)

| | Solvent | Danshen | | | |
|---|---|---|---|---|---|
| | control | Tablet group | TSG | TSG | TSG |
| | group | 45 mg/kg | 5 mg/kg | 10 mg/kg | 20 mg/kg |
| LDH | 206 ± 47.3 | 173 ± 81.4 | 159 ± 80.4 | 140 ± | 120 ± |
| | | | | 47.6** | 40.0** |

**P < 0.01 vs solvent control group

(6) Effect of TSG on area of acute myocardial infarction in anesthetized dogs: Quantitative histological detection (N-BT staining method) demonstrated that in comparison with solvent control group, the areas of myocardial infarction minimized significantly in Danshen Tablet group and all TSG groups (See Table 12).

TABLE 12
Effect of oral TSG on area of acute myocardial infarction in anesthetized dogs
( x̅ ± s, g, n = 6)

| | Solvent control | Danshen Tablet group | TSG | TSG | TSG |
|---|---|---|---|---|---|
| | group | 45 mg/kg | 5 mg/kg | 10 mg/kg | 20 mg/kg |
| Weight of heart (g) | 68.3 ± 11.6 | 78.7 ± 15.6 | 79.7 ± 6.68 | 68.8 ± 6.05 | 80.6 ± 13.3 |
| weight of left heart (g) | 46.2 ± 9.26 | 53.8 ± 10.8 | 55.1 ± 4.15 | 48.2 ± 4.84 | 56.2 ± 9.58 |
| weight of infarct myocardium (g) | 4.58 ± 0.83 | 3.67 ± 0.98* | 5.98 ± 1.71 | 3.17 ± 0.66* | 2.31 ± 0.70** |
| Weight of IM/Weight of LH | 9.96 ± 1.68 | 6.94 ± 1.67* | 10.8 ± 2.81 | 6.61 ± 1.31** | 4.03 ± 0.61*** |

*P < 0.05,
**P < 0.01,
***P < 0.001 vs solvent control group

### Conclusion

(1) After intragastric administration at the doses of 5, 10 and 20 mg/kg, 3,4′,5-trihydroxy-stilbene-3-β-D-glucoside can reduce the increment of ST segment elevation in surface ECG and epicardial electrogram of canine myocardial ischemia induced by coronary artery ligation, demonstrating that oral administration of the chemical has the effect to relieve myocardial ischemia.

(2) With method of N-BT staining, it is demonstrated that oral administration of 3,4′,5-trihydroxy-stilbene-3-β-D-glucoside at the doses of 10 and 20 mg/kg may significantly reduce the extent of acute myocardial ischemia and minimize the area of myocardial infarction in anesthetized dogs. Detection of LDH activity proves that oral administration of 3,4′,5-trihydroxy-stilbene-3-β-D-glucoside at the doses of 5, 10 and 20 mg/kg can dose dependently inhibit LDH level rise after acute myocardial infarction in dogs, indicating that oral administration of 3,4′,5-trihydroxy-stilbene-3-β-D-glucoside has protective action against myocardial injury induced by acute myocardial infarction in anesthetized dogs.

### EXAMPLE 5

### Effect of 3,4′,5-trihydroxy-stilbene-3-β-D-glucoside iv on Myocardial Oxygen Consumption in Anesthetized Dogs

The purpose of the present example is to observe intravenous administration of 3,4′,5-trihydroxy-stilbene-3-β-D-glucoside on myocardial oxygen consumption in anesthetized dogs.

### Test Drugs

Test drug: 3,4′,5-trihydroxy-stilbene-3-□-D-glucoside (Batch no: 03030302), 100 mg/10 ml; diluted with normal saline before use.

Control drug: sorbide nitrate injection (Isosorbide Dinitrate, batch no: 479210) manufactured by Germany Schwarz Pharma AG and repacked by Zhuhai Schwarz Pharma Co Ltd.

### Test Animal

Healthy male and female hybrid dogs with each weight 10˜14 kg, provided by experimental animal center in First Military Medical University.

### Groups and Administration

Solvent control group, Isosorbide Dinitrate control group (0.4 mg/kg/h) and administration groups of 3,4′,5-trihydroxy-stilbene-3-β-D-glucoside at low (2.5 mg/kg), middle (5 mg/kg) or high (10 mg/kg) doses were set up for the test. Isosorbide Dinitrate control group were medicated by continuous intravenous infusion and other groups by intravenous injection.

### Test Method

The animals were anesthetised with 3% pentobarbital sodium iv at the dose of 30 mg/kg. Tracheal intubation was performed and SC-M5 anesthesia respirator (manufactured by Shanghai Medical Instrument Factory, frequency: 16˜18 per min, tidal volume: 350˜550 ml) was connected. Bilateral femoral arteries were separated for blood sampling and analyzing and measurement of mean blood pressure. Thoracotomy at the fourth intercostal space was performed to expose the heart and to open the pericardium to make pericardial hammock. Ascending aortic root and upper part of left branch of anterior descending coronary artery were dissociated to put respectively probes of electromagnetic flowmeter (MFV-1100/1200, made by Japanese Nihon Kohden Company) to measure cardiac output and coronary artery flow rate. Right jugular vein was separated for insertion of a cardiac catheter that was guided into coronary venous sinuses before fixation. Synchronously, blood was sampled by drawing from coronary sinus and femoral artery (0.5% heparin was added for anticoagulation). pO2 and pH were detected with a blood gas analyzer (DH-1830 Blood Gas Acid-base Analyze, Nanjing Ananalysis Apparatus Factor) and the values were converted to artery and venous oxygen content. After intravenous infusion of control solvent or the drug, constant infusion for all groups was completed with an electronic constant flow pump (SH-88AB Controllable Intravenous Injector made by Quanzhou Lizhong Electronic Medical Instrument Factory). Both artery and venous blood gas before administration or 5, 15, 30, 60, 90 and 120 min after administration was analyzed and mean blood pressure and cardiac output at 0, 5, 15, 30, 60, 90 and 120 min were observed. At the end of experiment, the weight of heart was measured and the blackened part measured with the electromagnetic flowmeter by adverse infusion of 10-15 ml of 10% Shanghai Superior Carbon Ink was cut off and weighted for calculation of myocardial oxygen consumption and the indexes of myocardial oxygen consumption before and 5, 15, 30, 60, 90 and 120 min after administration with the formula as follows.

Myocardial oxygen consumption(ml/min/100 g)=coronary flow ml/min/100 g×artery oxygen−coronary sinus oxygen ml %).

Myocardial oxygen uptake (%)=(artery oxygen ml %−coronary sinus oxygen ml %)/artery oxygen ml %

Total peripheral resistance of general circulation(dyn·s·cm−5)=mean arterial blood pressure[MAP(KPa)]×80/cardiac output[CO(L/min)]=mean arterial blood pressure[MAP(mmHg)]×10.6/cardiac output[CO(L/min)]

Coronary resistance[Kpa/ml/min]=mean arterial blood pressure[MAP(KPa)]/coronary artery flow.

### Test Results

(1) Effect on myocardial oxygen consumption in anesthetized dogs: After intravenous injection of 3,4′,5-trihydroxy-stilbene-3-β-D-glucoside (TSG) at the doses of 2.5, 5, 10 mg/kg, myocardial oxygen consumption exhibited no significant difference in comparison with solvent control group (P>0.05). See Table 13 for details.

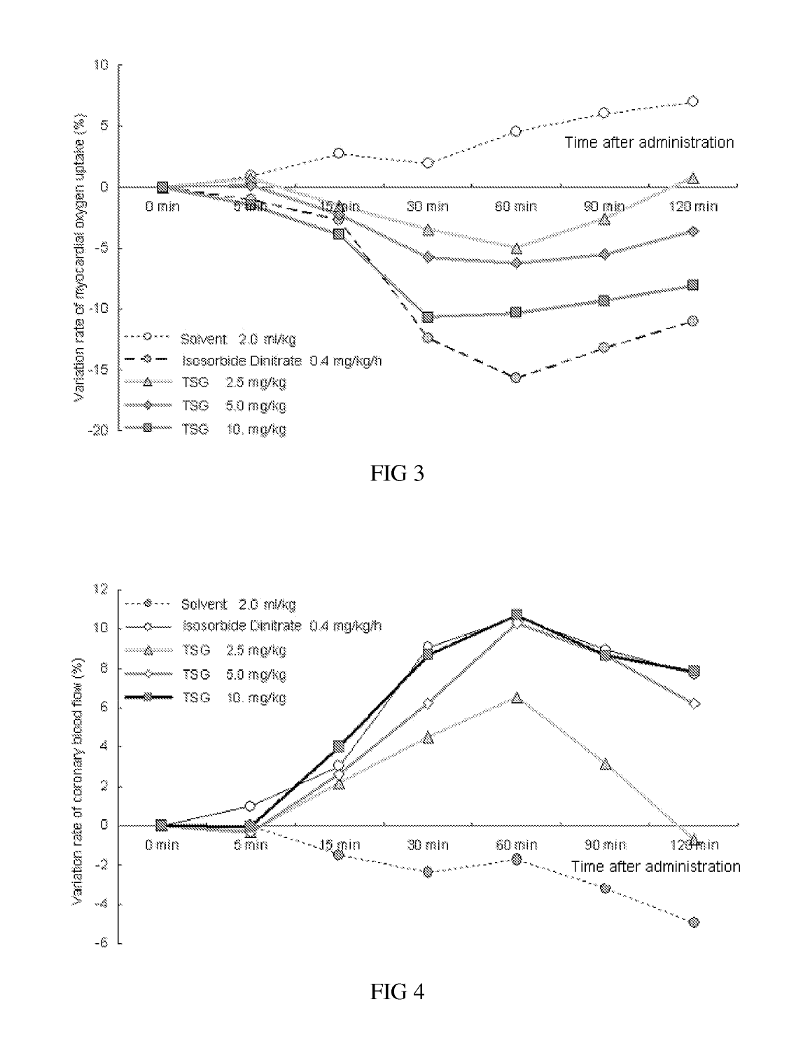

(2) Effect on myocardial oxygen uptake in anesthetized dogs: After intravenous injection of 3,4′,5-trihydroxy-stilbene-3-β-D-glucoside (TSG), the myocardial oxygen uptake was decreased remarkably and the differences of rates of reduction were significant in low dose group at 15, 60 and 90 min when compared with solvent control group at the same time points. In middle and high groups, the differences of myocardial oxygen uptake reduction were of great significant at the time from 15 min to 120 min in comparison with solvent control group. See Table 14 and FIG. 3 for details.

(3) Effect on coronary flow rate in anesthetized dogs: Coronary flow rates were increased in all dose group of 3,4′,5-trihydroxy-stilbene-3-β-D-glucoside (TSG). In comparison with solvent control group at the same time points, the rates of increment in low dose group differed significantly from 15 min on (P<0.05) and differed very significantly between 30˜90 min (P<0.01). In middle and high dose groups, differences were very significant during the time period between 15˜120 min. See Table 15 and FIG. 4 for details.

(4) Effect on coronary artery resistance: In all dose groups, TSG intravenous injection made no significant difference on coronary artery resistance at the time between 5˜90 min, but the rate of reduction of coronary artery resistance in middle and high dose groups differed very significantly at 120 min in comparison with solvent control group at the same time point (P<0.01). See Table 16 for details.

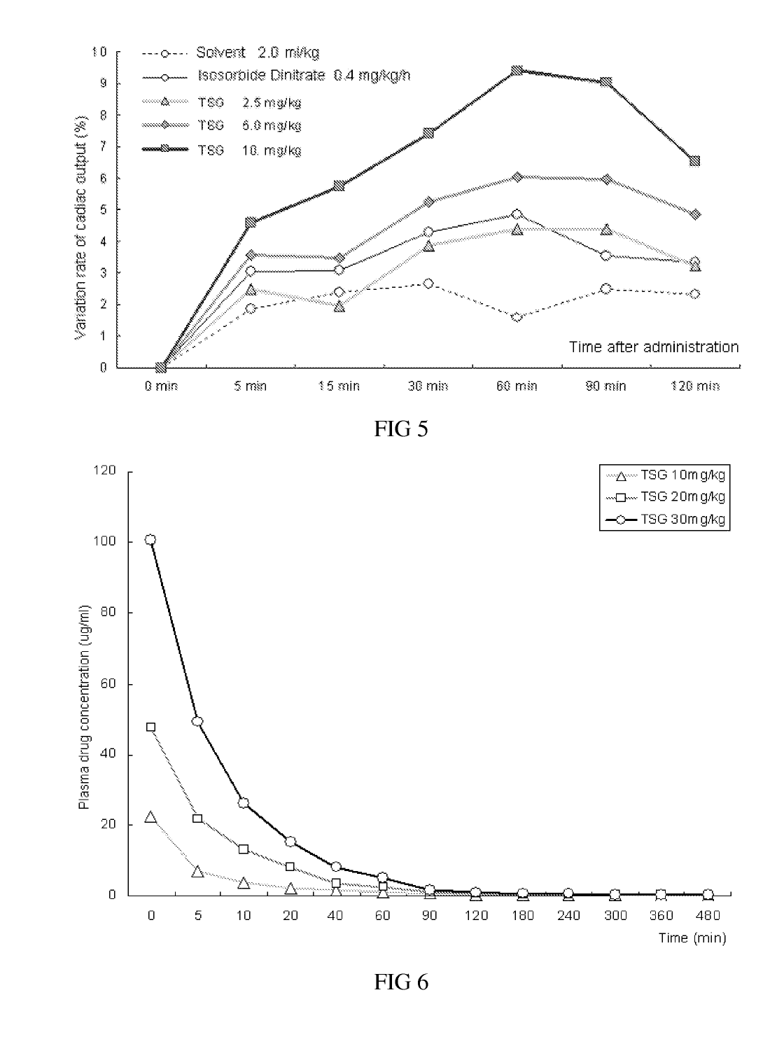

(5) Effect on cardiac output and peripheral resistance in anesthetized dogs: TSG could increase the cardiac output in dogs. In comparison with solvent control group at the same time points, no significant difference was witnessed at 60 and 90 min in middle dose group (P<0.05) and at 15 and 30 min in high dose group (P<0.01), but very great differences could be observed at 60 and 90 min in high dose group (P<0.01). Positive control group got no influence on their cardiac output. TSG had no effect on peripheral resistance. See Table 17, 18 and FIG. 5 for details.

### Conclusion

3,4′,5-trihydroxy-stilbene-3-β-D-glucoside can obviously increase coronary artery flow, and cardiac output in anesthetized dogs and decrease their myocardial oxygen uptake and the resistance of coronary artery. It has no obvious effect on myocardial oxygen consumption and peripheral resistance.

TABLE 13
Effect of TSG iv on canine myocardial oxygen consumption
(mL/min.100 g⁻¹, x̅ ± s, n = 6)

| | | before | | | | | | |
|---|---|---|---|---|---|---|---|---|
| | | administration | After administration | | | | | |
| Group | Index | 0 min | 5 min | 15 min | 30 min | 60 min | 90 min | 120 min |
| Solvent control | x̅ ± s | 214.6 ± 7.34 | 216.2 ± 7.51 | 217.0 ± 6.78 | 214.4 ± 7.48 | 217.3 ± 4.87 | 219.4 ± 7.65 | 217.0 ± 6.38 |
| 2 ml/kg | variation % | | 0.73 ± 2.15 | 1.15 ± 3.68 | −0.10 ± 2.28 | 1.31 ± 3.25 | 2.29 ± 4.98 | 1.1 ± 1.67 |
| Isosorbide | x̅ ± s | 212.8 ± 16.2 | 209.6 ± 10.5 | 212.9 ± 12.9 | 202.0 ± 11.8* | 196.0 ± 5.52** | 199.3 ± 6.67** | 200.5 ± 16.0 |
| Dinitrate 0.4 | variation % | | −1.46 ± 9.67 | 0.10 ± 7.99 | −5.0 ± 19.1* | −7.83 ± 1.67** | −6.3 ± 2.38** | −5.6 ± 6.4 |
| mg/kg/h | | | | | | | | |
| TSG 2.5 mg/kg | x̅ ± s | 214.6 ± 5.24 | 215.5 ± 8.73 | 217.0 ± 6.75 | 218.2 ± 6.85 | 218.0 ± 8.95 | 214.1 ± 8.39 | 214.0 ± 6.87 |
| | variation % | | 0.40 ± 3.16 | 1.11 ± 1.44 | 1.66 ± 1.48 | 1.57 ± 2.94 | −0.25 ± 2.84 | −0.3 ± 1.9 |
| TSG 5 mg/kg | x̅ ± s | 211.4 ± 4.05 | 210.4 ± 6.48 | 211.5 ± 9.81 | 210.2 ± 8.65 | 212.9 ± 7.2 | 209.6 ± 9.01 | 211.7 ± 8.07 |
| | variation % | | −0.52 ± 1.59 | 0.05 ± 4.29 | −0.59 ± 2.68 | 0.68 ± 1.74 | −0.91 ± 2.81 | 0.14 ± 4.0 |
| TSG 10 mg/kg | x̅ ± s | 214.1 ± 6.66 | 211.0 ± 5.44 | 215.0 ± 3.39 | 208.0 ± 5.3 | 210.3 ± 6.32 | 209.1 ± 6.38 | 208.7 ± 7.78 |
| | variation % | | −1.45 ± 1.13 | 0.44 ± 2.01 | −2.77 ± 3.75 | −1.75 ± 3.51 | −2.28 ± 3.5 | −2.5 ± 4.33 |

*P < 0.05,
**P < 0.01 in comparison with solvent control group

TABLE 14
Effect of TSG iv on myocardial oxygen uptake (%, x̅ ± s, n = 6)

| | | before | | | | | | |
|---|---|---|---|---|---|---|---|---|
| | | administration | After administration | | | | | |
| Group | Index | 0 min | 5 min | 15 min | 30 min | 60 min | 90 min | 120 min |
| Solvent | x̅ ± s | 79.1 ± 3.53 | 79.8 ± | 81.3 ± 4.09 | 80.7 ± 3.60 | 82.7 ± 3.61 | 83.9 ± 4.25 | 84.7 ± 4.06 |
| control | | | 3.46 | | | | | |
| 2 ml/kg | variation % | | 0.9 ± | 2.75 ± 1.96 | 1.96 ± 1.58 | 4.57 ± 3.04 | 6.04 ± 3.35 | 7.01 ± 2.70 |
| | | | 2.29 | | | | | |
| Isosorbide | x̅ ± s | 75.4 ± 4.31 | 74.6 ± | 73.3 ± 3.59* | 66.0 ± 3.69 | 63.6 ± 3.92** | 65.5 ± 4.03** | 67.3 ± 9.07** |
| Dinitrate 0.4 | | | 2.99 | | | | | |
| mg/kg/h | variation % | | −1.02 ± | −2.71 ± 1.08** | −12.4 ± 1.93** | −15.7 ± 1.68** | −13.2 ± 2.22** | −11.0 ± 8.2** |
| | | | 2.44 | | | | | |
| TSG 2.5 | x̅ ± s | 76.9 ± 5.50 | 77.4 ± | 75.7 ± 5.53 | 74.2 ± 5.11 | 73.1 ± 6.16 | 75.0 ± 6.24 | 77.5 ± 5.23 |
| mg/kg | | | 5.38 | | | | | |
| | variation % | | 0.72 ± | −1.55 ± 1.97* | −3.47 ± 1.59 | −4.99 ± 3.01** | −2.58 ± 2.67** | 0.78 ± 3.24 |
| | | | 1.72 | | | | | |
| TSG 5 | x̅ ± s | 79.5 ± 6.93 | 79.6 ± | 77.5 ± 4.22 | 74.8 ± 5.66 | 74.5 ± 6.05 | 75.1 ± 6.48 | 76.6 ± 7.07 |
| mg/kg | | | 5.92 | | | | | |
| | variation % | | 0.15 ± | −2.26 ± 3.95** | −5.76 ± 5.34** | −6.24 ± 4.39** | −5.51 ± 4.5** | −3.65 ± 4.29** |
| | | | 1.81 | | | | | |
| TSG 10 | x̅ ± s | 80.0 ± 6.55 | 78.7 ± | 76.8 ± 5.70 | 71.3 ± 5.28 | 71.7 ± 5.71* | 72.4 ± 5.51** | 73.4 ± 5.24* |
| mg/kg | | | 5.83 | | | | | |
| | variation % | | −1.46 ± | −3.89 ± 1.18** | −10.7 ± 4.01** | −10.3 ± 5.25** | −9.32 ± 3.22** | −8.1 ± 3.21** |
| | | | 3.92 | | | | | |

*P < 0.05,
**P < 0.01 in comparison with solvent control group

TABLE 15
Effect of TSG on coronary artery flow (ml/min, x̅ ± s, n = 6)

| | | before | | | | | | |
|---|---|---|---|---|---|---|---|---|
| | | administration | After administration | | | | | |
| Group | Index | 0 min | 5 min | 15 min | 30 min | 60 min | 90 min | 120 min |
| Solvent | x̅ ± s | 42.2 ± 5.81 | 42.2 ± | 41.5 ± 5.56 | 41.2 ± 5.73 | 41.3 ± 4.96 | 40.8 ± 5.84 | 40.0 ± 4.93 |
| control | | | 5.81 | | | | | |
| 2 ml/kg | variation % | | 0.0 ± | −1.52 ± 1.09 | −2.39 ± 1.19 | −1.73 ± 2.11 | −3.2 ± 1.81 | −4.94 ± 1.86 |
| | | | 0.0 | | | | | |
| Isosorbide | x̅ ± s | 43.5 ± 7.26 | 43.8 ± | 44.8 ± 7.65 | 43.3 ± 7.17 | 48.0 ± 7.51 | 47.3 ± 7.55 | 46.8 ± 7.89 |
| Dinitrate 0.4 | | | 6.80 | | | | | |
| mg/kg/h | variation % | | 0.96 ± | 3.02 ± 1.51** | 9.08 ± 2.45** | 10.6 ± 2.96** | 8.96 ± 2.96** | 7.72 ± 4.14** |
| | | | 1.91 | | | | | |
| TSG 2.5 | x̅ ± s | 44.2 ± 7.57 | 44.0 ± | 45.2 ± 8.04 | 46.2 ± 8.01 | 47.0 ± 7.87 | 45.5 ± 7.40 | 43.8 ± 7.36 |
| mg/kg | | | 7.32 | | | | | |
| | variation % | | −0.31 ± | 2.16 ± 1.27* | 4.51 ± 1.17** | 6.52 ± 1.80** | 3.15 ± 1.46** | −0.70 ± 1.09 |
| | | | 0.76 | | | | | |
| TSG 5 | x̅ ± s | 40.8 ± 5.85 | 40.7 ± | 41.8 ± 5.42 | 43.3 ± 5.82 | 45.0 ± 6.07 | 44.3 ± 6.09 | 43.3 ± 5.92 |
| mg/kg | | | 5.50 | | | | | |
| | variation % | | −0.33 ± | 2.6 ± 1.68** | 6.23 ± 2.19** | 10.3 ± 1.97** | 8.65 ± 2.56** | 6.21 ± 2.78** |
| | | | 0.80 | | | | | |
| TSG 10 | x̅ ± s | 44.0 ± 5.83 | 44.0 ± | 45.7 ± 5.28 | 47.8 ± 6.46 | 48.7 ± 6.19 | 47.8 ± 6.71 | 47.5 ± 6.83 |
| mg/kg | | | 6.03 | | | | | |
| | variation % | | −0.05 ± | 4.01 ± 2.75** | 8.68 ± 0.97** | 10.7 ± 3.53** | 8.65 ± 3.62** | 7.85 ± 3.77** |
| | | | 2.50 | | | | | |

*P < 0.05,
**P < 0.01 in comparison with solvent control group

TABLE 16
Effect of TSG on coronary artery resistance (Kpa/ml/min, x̅ ± s, n = 6)

| | | before | | | | | | |
|---|---|---|---|---|---|---|---|---|
| | | administration | After administration | | | | | |
| Group | Index | 0 min | 5 min | 15 min | 30 min | 60 min | 90 min | 120 min |
| Solvent | x̅ ± s | 0.271 ± 0.024 | 0.275 ± | 0.275 ± 0.037 | 0.282 ± 0.030 | 0.276 ± 0.023 | 0.281 ± 0.026 | 0.288 ± 0.020 |
| control | | | 0.032 | | | | | |
| 2 ml/kg | variation % | | 1.36 ± | 1.24 ± 9.29 | 3.99 ± 4.30 | 2.06 ± 2.82 | 3.68 ± 1.61 | 6.31 ± 3.52 |
| | | | 7.11 | | | | | |
| Isosorbide | x̅ ± s | 0.295 ± 0.016 | 0.280 ± | 0.272 ± 0.022 | 0.253 ± 0.016 | 0.248 ± 0.020 | 0.254 ± 0.017 | 0.267 ± 0.027 |
| Dinitrate | | | 0.025 | | | | | |
| 0.4 mg/kg/h | variation % | | −5.00 ± | −7.91 ± 3.24** | −14.28 ± 1.96** | −15.97 ± 2.66** | −13.75 ± 0.99** | −9.71 ± 4.80** |
| | | | 3.71 | | | | | |
| TSG | x̅ ± s | 0.268 ± 0.050 | 0.269 ± | 0.266 ± 0.049 | 0.257 ± 0.054 | 0.261 ± 0.051 | 0.267 ± 0.054 | 0.271 ± 0.055 |
| 2.5 mg/kg | | | 0.050 | | | | | |
| | variation % | | 0.81 ± | −0.34 ± 2.36 | −3.82 ± 9.83 | −2.48 ± 8.17 | −0.24 ± 10.72 | 1.19 ± 6.55 |
| | | | 6.09 | | | | | |
| TSG | x̅ ± s | 0.299 ± 0.029 | 0.308 ± | 0.305 ± 0.029 | 0.299 ± 0.026 | 0.288 ± 0.026 | 0.287 ± 0.028 | 0.287 ± 0.031 |
| 5 mg/kg | | | 0.028 | | | | | |
| | variation % | | 3.03 ± | 2.18 ± 2.46 | 0.02 ± 3.86 | −3.62 ± 3.63 | −4.03 ± 2.13 | −3.99 ± 3.34** |
| | | | 1.60 | | | | | |
| TSG | x̅ ± s | 0.273 ± 0.032 | 0.286 ± | 0.278 ± 0.028 | 0.274 ± 0.035 | 0.267 ± 0.037 | 0.270 ± 0.040 | 0.263 ± 0.038 |
| 10 mg/kg | | | 0.036 | | | | | |
| | variation % | | 4.63 ± | 1.79 ± 3.25 | 0.21 ± 1.44 | −2.64 ± 2.78 | −1.69 ± 4.78 | −4.10 ± 5.44** |
| | | | 3.07 | | | | | |

*P < 0.05,
**P < 0.01 in comparison with solvent control group

TABLE 17
Effect of TSG on canine cardiac output (L/min, x̅ ± s, n = 6)

| | | before | | | | | | |
|---|---|---|---|---|---|---|---|---|
| | | administration | After administration | | | | | |
| Group | Index | 0 min | 5 min | 15 min | 30 min | 60 min | 90 min | 120 min |
| Solvent control | x̅ ± s | 1.09 ± 0.12 | 1.11 ± 0.12 | 1.11 ± 0.12 | 1.12 ± 0.12 | 1.11 ± 0.14 | 1.11 ± 0.12 | 1.11 ± 0.11 |
| 2 ml/kg | variation % | | 1.88 ± 0.96 | 2.38 ± 2.08 | 2.65 ± 1.45 | 1.59 ± 1.82 | 2.49 ± 0.82 | 2.33 ± 3.46 |
| Isosorbide | x̅ ± s | 1.11 ± 0.12 | 1.14 ± 0.12 | 0.14 ± 0.11 | 0.15 ± 0.11 | 1.16 ± 0.12 | 0.14 ± 0.11 | 1.14 ± 0.12 |
| Dinitrate | variation % | | 3.05 ± 2.86 | 3.08 ± 1.75 | 4.31 ± 1.87 | 4.86 ± 2.26 | 3.55 ± 2.82 | 3.36 ± 1.94 |
| 0.4 mg/kg/h | | | | | | | | |
| TSG | x̅ ± s | 1.06 ± 0.13 | 1.09 ± 0.12 | 1.09 ± 0.14 | 1.10 ± 0.13 | 1.11 ± 0.13 | 1.11 ± 0.13 | 1.10 ± 0.14 |
| 2.5 mg/kg | variation % | | 2.50 ± 2.42 | 1.97 ± 1.55 | 3.88 ± 3.48 | 4.41 ± 1.40 | 4.40 ± 1.64 | 3.23 ± 2.57 |
| TSG | x̅ ± s | 1.09 ± 0.14 | 1.13 ± 0.12 | 1.13 ± 0.13 | 1.15 ± 0.14 | 1.16 ± 0.14 | 1.16 ± 0.13 | 1.14 ± 0.13 |
| 5 mg/kg | variation % | | 3.58 ± 2.00 | 3.47 ± 0.97 | 5.25 ± 2.62 | 6.05 ± 2.06* | 5.98 ± 2.02* | 4.86 ± 2.88 |
| TSG | x̅ ± s | 1.08 ± 0.15 | 1.13 ± 0.13 | 1.14 ± 0.14 | 1.16 ± 0.13 | 1.18 ± 0.14 | 1.18 ± 0.15 | 1.15 ± 0.16 |
| 10 mg/kg | variation % | | 4.60 ± 3.19 | 5.74 ± 2.02* | 7.43 ± 3.40* | 9.41 ± 4.82** | 9.05 ± 2.24** | 6.53 ± 2.43* |

*P < 0.05,
**P < 0.01 in comparison with solvent control group

TABLE 18
Effect of TSG on total peripheral resistance (TPR, dyn·s·cm⁻⁵, x̅ ± s, n = 6)

| | | before | | | | | | |
|---|---|---|---|---|---|---|---|---|
| | | administration | After administration | | | | | |
| Group | Index | 0 min | 5 min | 15 min | 30 min | 60 min | 90 min | 120 min |
| Solvent | x̅ ± s | 845.3 ± 146.3 | 841.7 ± 160.9 | 815.0 ± 96.0 | 832.0 ± 117.6 | 834.5 ± 149.2 | 830.3 ± 166.7 | 830.5 ± 109.8 |
| control | variation % | | −0.50 ± 7.08 | −2.56 ± 9.55 | −1.07 ± 5.41 | −1.26 ± 3.04 | −2.06 ± 3.27 | −1.16 ± 4.82 |
| 2 ml/kg | | | | | | | | |
| Isosorbide | x̅ ± s | 924.4 ± 109.9 | 860.7 ± 116.3 | 848.7 ± 73.6 | 827.7 ± 99.6 | 818.7 ± 107.2 | 837.9 ± 87.9 | 868.8 ± 113.3 |
| Dinitrate | variation % | | −6.88 ± 5.39 | −7.97 ± 2.92 | −10.35 ± 0.76* | −11.43 ± 1.49** | −9.19 ± 3.65 | −6.05 ± 1.79 |
| 0.4 mg/kg/h | | | | | | | | |
| TSG | x̅ ± s | 888.8 ± 200.9 | 873.5 ± 210.3 | 888.7 ± 208.8 | 869.1 ± 249.3 | 890.2 ± 240.3 | 877.6 ± 225.1 | 868.6 ± 221.8 |
| 2.5 mg/kg | variation % | | −1.89 ± 6.74 | −0.10 ± 4.21 | −3.22 ± 9.65 | −0.56 ± 7.52 | −1.52 ± 9.42 | −2.58 ± 7.26 |
| TSG | x̅ ± s | 906.3 ± 193.9 | 897.1 ± 184.7 | 916.6 ± 186.2 | 915.2 ± 199.9 | 907.8 ± 194.0 | 891.7 ± 188.6 | 881.8 ± 188.9 |
| 5 mg/kg | variation % | | −0.82 ± 2.72 | 1.31 ± 2.04 | 0.90 ± 1.20 | 0.20 ± 1.19 | −1.59 ± 3.63 | −2.75 ± 3.55 |
| TSG | x̅ ± s | 891.8 ± 93.4 | 890.5 ± 82.2 | 891.3 ± 78.1 | 903.4 ± 76.9 | 878.7 ± 90.9 | 872.9 ± 98.6 | 863.7 ± 80.0 |
| 10 mg/kg | variation % | | 0.02 ± 4.07 | 0.09 ± 2.25 | 1.49 ± 3.81 | −1.42 ± 3.68 | −2.16 ± 2.50 | −3.00 ± 4.04 |

*P < 0.05,
**P < 0.01 in comparison with solvent control group

### EXAMPLE 6

### Effect of 3,4′,5-trihydroxy-stilbene-3-β-D-glucoside iv on Hemodynamics in Anesthetized Dogs

The purpose of the example is to test and observe the effect of 3,4′,5-trihydroxy-stilbene-3-β-D-glucoside iv administration on hemodynamic indexes such as heart rate, blood pressure and systolic and diastolic function of heart.

### Test Drugs

Test drug: 3,4′,5-trihydroxy-stilbene-3-β-D-glucoside (Batch no: 03030302, provided by Shenzhen Neptunus Pharmaceutical Co., Ltd.), 100 mg/10 ml; diluted with normal saline before use.

Control drug: Sorbide nitrate injection (Isosorbide Dinitrate, batch no: 479210) manufactured by Germany Schwarz Pharma AG and repacked by Zhuhai Schwarz Pharma Co., Ltd.

### Test Animal

Healthy male and female hybrid dogs with each weight 10˜14 kg, provided by experimental animal center in First Military Medical University.

### Groups and Administration

Solvent control group, Isosorbide Dinitrate control group (0.4 mg/kg/h) and administration groups of 3,4′,5-trihydroxy-stilbene-3-□-D-glucoside at low (2.5 mg/kg), middle (5 mg/kg) or high (10 mg/kg) doses were set up for the test. Isosorbide Dinitrate control group were medicated by continuous intravenous infusion and other groups by intravenous injection.

### Test Method

After anesthetized with pentobarbital sodium, the dog was immobilized on its back. A femoral artery was separated for insertion of a catheter to record blood pressure of artery. A cardiac catheter was guided inversely from right common carotid artery into its left ventricle. Both catheters were connected to pressure transmitters. After pressure signals were amplified with carriers, they were input into an electrophysiolograph Powerlab system 8s, (ML785/8S AD Instruments, Australia). Needle electrodes were punctured into four limbs to monitor standard 2-lead ECG. A computer (chart4.12, ML785/8S, AD Instruments, Australia) were applied for real-time monitoring and storage of data. After operation, there were 30 minutes for stableness and then basic indexes were recorded before administration. Negative control group were given by extraneously injection of solvent at 2 ml/kg, while positive control group were intravenously infused at 0.4 mg/kg/h. For administration groups, TSG were given by intravenously injection with an electronic constant flow pump (SH-88AB Controllable Intravenous Injector made by Quanzhou Lizhong Electronic Medical Instrument Factory) and the constant infusion was finished with 30 minutes. Indexes were recorded at the time points of 10, 30, 60 and 120 min after administration.

Indexes of observation included heart rate (HR), systolic blood pressure (BPs), diastolic blood pressure, mean blood pressure, left ventricular systolic pressure (LVSP), left ventricular diastolic pressure (LVDP), the maximum change rate of left ventricular systolic pressure rise and fall (±dp/dtmax), left ventricular end diastolic pressure and ECG.

Test results were demonstrated with x̅±s and SPSS 10.0 software was used for analysis of variance. Significance of differences was checked.

### Test Results

Results showed: after intravenous injection of 3,4′,5-trihydroxy-stilbene-3-□-D-glucoside (TSG) at the doses of 2.5 mg/kg and 5 mg/kg, heart rate, blood pressure, left ventricular pressure and the maximum change rate of left ventricular systolic pressure rise and fall fluctuated, but with no significant difference in comparison with those before administration. TSG at the dose of 10 mg/kg iv increased blood pressure in anesthetized dogs, but with no significant difference in comparison with that before administration. Other indexes presented also no obvious changes. See Table 19 to 23 for details.

TABLE 19
Effect of solvent at 2 ml/kg iv on hemodynamics of anesthetized dogs
( x̅ ± s, n = 5)

| | Time | | | | |
|---|---|---|---|---|---|
| | 0 | 10 | 30 | 60 | 120 |
| HR (p/min) | 169.5 ± 29.5 | 171.8 ± 28.4 | 170.6 ± 24.9 | 179.7 ± 42.6 | 168.0 ± 32.4 |
| BPs (KPa) | 23.0 ± 3.9 | 22.6 ± 3.3 | 23.1 ± 3.0 | 24.2 ± 2.8 | 23.3 ± 2.2 |
| BPm (KPa) | 19.0 ± 3.7 | 19.0 ± 2.9 | 19.5 ± 3.0 | 20.5 ± 2.8 | 19.6 ± 2.1 |
| BPd (KPa) | 16.7 ± 3.4 | 16.6 ± 2.6 | 17.2 ± 3.1 | 18.1 ± 3.1 | 17.1 ± 2.3 |
| LVSP(KPa) | 23.5 ± 4.5 | 24.4 ± 3.8 | 24.7 ± 3.8 | 23.1 ± 5.8 | 23.5 ± 6.5 |
| LVDP (KPa) | 0.24 ± 1.49 | 0.34 ± 1.58 | 0.45 ± 1.44 | 0.34 ± 1.44 | 0.49 ± 1.59 |
| LVEDP(KPa) | 1.23 ± 1.70 | 1.11 ± 1.40 | 1.21 ± 1.31 | 1.00 ± 1.41 | 1.06 ± 1.66 |
| +dp/dt max (Kpa/s) | 801.1 ± 284.0 | 836.3 ± 185.7 | 731.4 ± 172.3 | 802.0 ± 334.0 | 847.1 ± 266.5 |
| −dp/dt max (Kpa/s) | 815.0 ± 105.3 | 733.1 ± 201.3 | 746.8 ± 124.3 | 759.4 ± 207.7 | 786.5 ± 180.0 |

TABLE 20
Effect of Isosorbide Dinitrate at 0.4 mg/kg/h iv on hemodynamics of anesthetized dogs
( x̅ ± s, n = 5)

| | Time | | | | |
|---|---|---|---|---|---|
| | 0 | 10 | 30 | 60 | 120 |
| HR(p/min) | 197.3 ± 36.5 | 198.6 ± 30.3 | 209.8 ± 31.7 | 204.7 ± 33.7 | 171.5 ± 34.6 |
| BPs (KPa) | 26.8 ± 1.2 | 25.3 ± 1.7 | 24.8 ± 1.3* | 24.9 ± 0.7* | 25.4 ± 1.0 |
| BPm (KPa) | 21.5 ± 0.7 | 20.6 ± 1.0 | 20.4 ± 0.8 | 20.5 ± 0.7 | 20.7 ± 0.8 |
| BPd (KPa) | 18.9 ± 0.6 | 18.4 ± 0.7 | 18.1 ± 0.7 | 18.2 ± 0.7 | 18.4 ± 0.8 |
| LVSP(KPa) | 24.1 ± 0.9 | 22.9 ± 1.3 | 23.0 ± 0.4 | 23.0 ± 0.8 | 22.7 ± 0.6 |
| LVDP (KPa) | −0.29 ± 0.42 | −0.37 ± 0.39 | −0.17 ± 0.34 | −0.42 ± 0.26 | −0.47 ± 0.21 |
| LVEDP(KPa) | 0.51 ± 0.13 | 0.58 ± 0.20 | 0.63 ± 0.16 | 0.39 ± 0.21 | 0.41 ± 0.08 |
| +dp/dt max (Kpa/s) | 1081.0 ± 334.0 | 1047.5 ± 266.3 | 1123.9 ± 225.3 | 1124.6 ± 228.4 | 972.5 ± 925.8 |
| −dp/dt max (Kpa/s) | 816.1 ± 121.2 | 786.5 ± 128.2 | 718.2 ± 103.5 | 751.1 ± 153.7 | 741.9 ± 102.4 |

TABLE 21
Effect of TSG at 2.5 mg/kg iv on hemodynamics of anesthetized dogs ( x̅ ± s, n = 5)

| | Time | | | | |
|---|---|---|---|---|---|
| | 0 | 10 | 30 | 60 | 120 |
| HR p/min) | 163.7 ± 21.1 | 160.4 ± 18.4 | 161.7 ± 18.0 | 171.1 ± 18.8 | 170.5 ± 16.1 |
| BPs (KPa) | 25.3 ± 3.0 | 24.5 ± 2.0 | 24.7 ± 1.3 | 25.5 ± 1.6 | 23.7 ± 3.3 |
| BPm (KPa) | 21.1 ± 2.0 | 20.7 ± 2.1 | 20.8 ± 1.7 | 21.6 ± 2.0 | 20.2 ± 2.3 |
| BPd (KPa) | 18.5 ± 1.7 | 18.3 ± 2.3 | 18.5 ± 2.2 | 19.1 ± 2.5 | 18.1 ± 1.8 |
| LVSP(KPa) | 24.9 ± 4.0 | 25.2 ± 4.2 | 25.7 ± 3.8 | 26.5 ± 2.5 | 26.7 ± 2.3 |
| LVDP (KPa) | 0.39 ± 2.20 | 0.45 ± 2.40 | 0.28 ± 2.40 | 0.26 ± 2.35 | 0.50 ± 2.06 |
| LVEDP(KPa) | 1.14 ± 2.18 | 1.00 ± 2.33 | 1.11 ± 2.50 | 0.78 ± 2.37 | 1.09 ± 2.16 |
| +dp/dt max (Kpa/s) | 853.1 ± 321.1 | 829.1 ± 308.3 | 869.6 ± 235.9 | 993.9 ± 206.2 | 957.9 ± 190.0 |
| −dp/dt max (Kpa/s) | 772.0 ± 201.0 | 777.3 ± 241.8 | 873.9 ± 236.8 | 905.9 ± 207.8 | 852.5 ± 159.6 |

TABLE 22
Effect of TSG at 5 mg/kg iv on hemodynamics of anesthetized dogs ( x̅ ± s, n = 5)

| | Time | | | | |
|---|---|---|---|---|---|
| | 0 | 15 | 30 | 60 | 120 |
| HR(p/min) | 177.2 ± 35.5 | 171.0 ± 35.6 | 170.0 ± 35.5 | 178.0 ± 29.5 | 170.2 ± 37.1 |
| BPs (KPa) | 25.4 ± 6.4 | 25.4 ± 5.8 | 26.1 ± 5.3 | 26.6 ± 4.1 | 25.0 ± 5.0 |
| BPm (KPa) | 21.1 ± 4.7 | 21.1 ± 4.7 | 21.7 ± 4.2 | 22.1 ± 3.3 | 21.0 ± 4.4 |
| BPd (KPa) | 18.9 ± 4.2 | 18.7 ± 4.4 | 19.2 ± 4.0 | 19.8 ± 3.3 | 18.8 ± 4.4 |
| LVSP(KPa) | 24.1 ± 4.4 | 24.3 ± 4.6 | 24.6 ± 4.6 | 25.6 ± 3.0 | 25.1 ± 5.2 |
| LVDP (KPa) | 0.31 ± 1.31 | −0.09 ± 2.03 | 0.18 ± 1.64 | 0.09 ± 1.29 | 0.27 ± 1.50 |
| LVEDP(KPa) | 1.05 ± 1.34 | 1.08 ± 1.62 | 1.19 ± 1.76 | 0.99 ± 1.19 | 0.98 ± 1.42 |
| +dp/dt max (Kpa/s) | 822.6 ± 353.8 | 793.8 ± 299.0 | 855.5 ± 296.5 | 936.1 ± 199.9 | 921.4 ± 327.1 |
| −dp/dt max (Kpa/s) | 866.2 ± 350.7 | 812.2 ± 308.9 | 912.3 ± 318.8 | 891.5 ± 261.8 | 872.0 ± 291.7 |

TABLE 23
Effect of TSG at 10 mg/kg iv on hemodynamics of anesthetized dogs ( x̅ ± s, n = 5)

| | Time | | | | |
|---|---|---|---|---|---|
| | 0 | 10 | 30 | 60 | 120 |
| HR(p/min) | 212.5 ± 31.3 | 209.7 ± 33.4 | 210.2 ± 33.8 | 207.1 ± 38.1 | 205.6 ± 50.2 |
| BPs (KPa) | 28.5 ± 7.5 | 32.4 ± 8.2 | 33.2 ± 8.5 | 33.6 ± 5.5 | 29.0 ± 4.8 |
| BPm (KPa) | 22.5 ± 4.8 | 24.5 ± 5.9 | 25.1 ± 5.9 | 24.4 ± 5.1 | 22.7 ± 3.0 |
| BPd (KPa) | 19.6 ± 3.7 | 21.0 ± 5.1 | 21.4 ± 5.5 | 20.6 ± 4.8 | 20.2 ± 3.0 |
| LVSP(KPa) | 30.1 ± 5.9 | 32.4 ± 6.3 | 32.4 ± 6.8 | 31.7 ± 5.5 | 29.5 ± 2.7 |
| LVDP (KPa) | −1.18 ± 1.71 | −1.30 ± 2.12 | −1.25 ± 2.47 | −1.87 ± 2.00 | −1.04 ± 2.11 |
| LVEDP(KPa) | 0.99 ± 0.60 | 0.93 ± 0.50 | 0.78 ± 0.43 | 0.63 ± 0.44 | 1.69 ± 2.78 |
| +dp/dt max (Kpa/s) | 1455.5 ± 637.7 | 1646.2 ± 807.8 | 1623.9 ± 729.5 | 1518.5 ± 708.1 | 1266.6 ± 334.8 |
| −dp/dt max (Kpa/s) | 1164.1 ± 488.8 | 1200.6 ± 447.6 | 1120.4 ± 357.8 | 1189.6 ± 461.6 | 1068.5 ± 208.6 |

### EXAMPLE 7

### Therapeutic Effect of Oral 3,4′,5-trihydroxy-stilbene-3-β-D-glucoside on Chronic Myocardial Ischemia in Rats

The purpose of the present example is to observe therapeutic effect of intragastric administration of 3,4′,5-trihydroxy-stilbene-3-β-D-glucoside on chronic myocardial ischemia in rats.

### Test Drug

3,4′,5-trihydroxy-stilbene-3-β-D-glucoside granules (Batch no: 031011), produced by Shenzhen Neptunus Pharmaceutical Co., Ltd, and prepared into a suspension with 0.8% CMC at the concentration of 2 and 4 mg/ml before use.

### Test Animal

Male SD rats with each weight 250˜300 g, provided by experimental animal center in First Military Medical University.

### Test Method

(1) Operation and Coronary artery ligation of Animal: 60 male SD rats, divided into two groups: 15 in sham operation group and 45 in operation group, were immobilized on their backs by anesthesia of urethane ip at the dose of 100 mg/kg. Electrocardiograph (ECG-6851C, made by Shanghai Photoelectrical & Electronic Medical Apparatus Co., Ltd.) was used to record II lead electrocardiogram. Tracheal intubation was performed and connected into an artificial respirator and thoracotomy was performed at the fourth intercostal space of left sternal border after the skin sterilization. With exposure of the heart, the ligation was conducted at 3-4 mm of the root of left anterior descending coronary artery. ECG was monitored during the whole process and the graph showing significant elevation and depression of ST segment as well as the elevated T-wave amplitude was viewed as the indexes for the success of coronary artery ligation. After the ligation, thorax was closed and the air was drawn out. Regular feeding was continued after the operation, and the intramuscular injection of Penicillin was performed to prevent infection in 1 week after the operation. Thoracotomy was performed in sham operation group without coronary artery ligation.

(2) Groups and administration: 5 weeks later, there were 11 rats survived in sham operation group, and 24 rats in operation group. Then the operation group was again randomized into two groups: ischemic control group and 3,4′,5-trihydroxy-stilbene-3-β-D-glucoside administrating group at the dose of 20 mg/kg. Starting from the sixth week, drugs were administered 6 days per week, and continued for 6 weeks, but withdrawn on Sundays. The volume of the drug administered was 5 ml/kg.

(1) Evaluation of therapeutic effect: Therapeutic effect was evaluated at the 12^{th} week after the operation. 30 min after the final administration, rats were immobilized on their backs and tracheal intubation was performed. Arteria Femoralis was separated or insertion of a catheter to record artery blood pressure. A cardiac catheter was inversely guided into left ventricular from right common carotid artery. Both catheters were connected with pressure sensor, and then the pressure signals were input into an electrophysiolograph Powerlab system 8s (ML785/8S, AD Instruments, made in Australia) after being amplified with carriers. Needle electrodes were subcutaneously inserted into four limbs to monitor the standard two lead ECG. A computer (chart4.12 software, ML785/8S, AD Instruments, made in Australia) was used to monitor and store data. Thirty min after the operation, heart rate (HR), blood pressure (BPm), left ventricular systolic pressure (LVSP), left ventricular diastolic pressure (LVDP), the maximal rate of left intraventricular pressure changes (+dp/dtmax), left ventricular end-diastolic pressure (LVEDP), ECG were observed. One hour later, animals were executed, and their left ventriculars were taken out. The left hearts were cut into slices of 5 mm in thickness, which were cleaned with normal saline and put into 0.025% nitroblue tetrazolium (N-BT, made by Swiss Fluka Chemistry Company) at 37° C. for staining. Normal myocardium was dyed into dark blue, and the myocardium in infarction area was not dyed, remaining still light-yellow. The infarction area was isolated from normal myocardium under an anatomic microscopy and the both weighed, separatively. The percentage (%) of the weight of infarct area to the weight of the whole was regarded as the index for measurement of the area of myocardial infarction.

Test results was expressed in x̅±s. Unpaired t-test was applied in statistic process, and there was a significant difference when P<0.05.

### Test Results

There was no significant difference of heart rate among sham operation group, ischemic control group and TSG administrating group. The mean artery blood pressure of ischemic control group was lower than that of sham operation group, while the artery blood pressure of TSG administrating group rose back to the level of sham operation group. The ECGs in both ischemic control group and TSG administrating group showed ST segment elevation or depression of different degrees, indicating the existence of myocardial ischemia. Compared to the control group, the left ventricular-developed pressure (LVDP) of ischemic control group increased, while its left ventricular end systolic pressure (LVESP) and ±dp/dtmax decreased. The corresponding hemodynamic indexes of TSG administrating group were much better, and the ischemic and infarcted tissue significantly diminished. The test results indicated that intragastric administration of 3,4′,5-trihydroxy-stilbene-3-β-D-glucoside (TSG) had significant therapeutic effect on chronic myocardial ischemia in rats (See Table 24).

TABLE 24
Therapeutic effect of TSG intragastric administration on rat
chronic myocardial ischemia ( x̅ ± s)

| | Sham | Ischemic | TSG |
|---|---|---|---|
| | operation | control | administrating |
| | group | group | group |
| n | 11 | 12 | 12 |
| HR (p/min) | 364 ± 32 | 383 ± 52 | 354 ± 45^{Δ} |
| BP (KPa) | 19.7 ± 1.2 | 17.5 ± 1.3* | 19.2 ± 1.8^{Δ} |
| LVSP(KPa) | 20.6 ± 1.8 | 17.7 ± 1.6* | 19.8 ± 2.1^{Δ} |
| LVEDP(KPa) | 0.68 ± 0.16 | 1.09 ± 0.25* | 0.82 ± 0.19^{Δ} |
| +dp/dtmax (Kpa/s) | 1456 ± 223 | 1106 ± 164* | 1389 ± 142^{Δ} |
| Infarct size ratio (%) | — | 14.5 ± 2.3 | 5.9 ± 1.2^{ΔΔ} |

*P < 0.05 vs sham operation group;
^{Δ}P < 0.05,
^{ΔΔ}P < 0.01 vs ischemic control group

### EXAMPLE 8

### Acute Toxicity Test of Mice Medicated by Intravenous Injection of 3,4′,5-trihydroxy-stilbene-3-β-D-glucoside

The purpose of the present example is to observe the acute toxicity reaction of mice after intravenous injection of 3,4′,5-trihydroxy-stilbene-3-β-D-glucoside.

3,4′,5-trihydroxy-stilbene-3-β-D-glucoside granules (Batch no: 031011), produced by Shenzhen Neptunus Pharmaceutical Co., Ltd, and diluted with normal saline before use.

### Test Animal

Kunming mice with each weight 18˜22 g, male and female in half, provided by experimental animal center in First Military Medical University

### Administration Method

Drugs were medicated via intravenous injection at different concentration in same volume, which was 20 ml/kg.

### Test Method

40 healthy Kunming mice were selected and randomly divided into 4 groups. Drugs were injected via tail vein at one time and the doses were 300 mg/kg, 425 mg/kg, 600 mg/kg and 850 mg/kg, respectively. Mice were constantly observed for 4 hours after the injection. After that, observations were carried out once in the morning and once in the afternoon for 7 days continuously. Signs of intoxation and time of death were recorded. Bliss method was applied to calculate LD50 and 95% confidence limits.

### Test Result

After the single dose of tail intravenous injection, body shaking, occasionally restlessness, twitch and other responses appeared. Those symptoms became more obvious when the dose was increased. The deaths of mice happened in 5 min to 24 h after the drug injection. Survivors recovered 2 days later, growing well with normal activities and euphagia. No abnormality was found during gross anatomy of main organs of dead mice. LD50 and 95% confidence limits are showed in Table 25.

TABLE 25
Results of acute toxicity test of mice medicated with TSG via
intravenous injection (n = 10)

| Dose | logarithm dose | Death | Death | LD₅₀ and 95% |
|---|---|---|---|---|
| (mg/kg) | (x) | number | rate (%) | confidence limits (g/kg) |
| 360 | 2.556 | 0 | 0 | LD₅₀ = 648.94 |
| 510 | 2.708 | 1 | 10 | 95% confidence limits |
| 720 | 2.857 | 7 | 70 | is 571.18~726.70 |
| 1020 | 3.009 | 10 | 100 | |

### Conclusion

LD50 of TSG medicated through mice intravenous injection is 648.94 mg/kg, the 95% confidence limit is 571.18 mg/kg˜726.70 mg/kg.

### EXAMPLE 9

### Pharmacokinetics of single-dose of 3,4′,5-trihydroxy-stilbene-3-β-D-glucoside by Intravenous Injection in Dogs

The purpose of the present example is to observe the pharmacokinetic parameters of single-dose of 3,4′,5-trihydroxy-stilbene-3-β-D-glucoside (TSG) iv in dogs.

### Test Drug

3,4′,5-trihydroxy-stilbene-3-β-D-glucoside solution (Batch no: 03030302, 100 mg/10 ml); produced by Shenzhen Neptunus Pharmaceutical CO., LTD.; diluted with normal saline before use.

### Test Animal

Healthy adult male and female Beagle dogs with each weight 10.8˜10.5 kg, provided by experimental animal center in Second Military Medical University

### Test Method

Pharmacokinetical studies were carried out in three groups at the doses of 10 mg/Kg, 20 mg/Kg and 30 mg/Kg. Five healthy adult Beagle dogs were chosen into each dose group. Tested Beagle dogs in each dose group were fasted for whole night (14 hours) before experiment, and were respectively intravenously injected with 10 mg/Kg, 20 mg/Kg and 30 mg/Kg of TSG in volume of 0.5 ml/Kg at 8:00 in the next morning. The intravenous injection was performed in one forelimb of each tested dog within 5 min in a very slow manner. Three hours after the intravenous injection, the dogs could be fed. 3 ml of venous blood was sampled in heparinized tubes from another forelimb of each tested dog before and 0, 5, 10, 20, 40, 60, 90, 120, 180, 240, 300, 360 and 480 min after administration. Blood samples were centrifuged to obtain 1 ml of plasma. TSG concentration in plasma was measured according to the method of blood sample pre-treatment (High concentration blood samples of Beagle dogs after the single-dose TSG injection should be diluted with blank plasma).

### Data Analysis

Non compartmental modeling method (statistical moment) was applied in the data analysis. Estimating formula of corresponding parameters is as follows:

AUC_{0˜τ}=Σ(Cᵢ+Cᵢ₋₁)×(tᵢ−tᵢ₋₁)/2

AUC0˜∞=Σ(Cᵢ+Cᵢ₋₁)×(tᵢ−tᵢ₋₁)/2+Cₙ/λ

AUMC0˜∞=Σ(Cᵢtᵢ+Cᵢ₋₁tᵢ₋₁)×(tᵢ−tᵢ₋₁)/2+Cₙ(1/λ²+tₙ/λ)

MRT=AUMC0˜∞/AUC0˜∞

Cl/F=D/AUC0˜∞

t_{1/2}=0.693/λ

V_{SS}=D×AUMC0˜∞/(AUC0˜∞)²

Where, in the formula, λ indicates the number of terminal eliminating rate of the concentration-time curve. tₙ and Cₙ represent the latest time of blood-sampling and plasma drug concentration, respectively. Pharmacokinetical parameters such as tmax and Cmax take the corresponding measuring value of plasma samples.

### Test Result

High-performance Liquid Chromatographic Method (HPLC) was used to measure the TSG plasma drug concentration in 5 healthy adult Beagle dogs in each of the administration groups at doses of 10 mg/Kg, 20 mg/Kg and 30 mg/Kgat different times. Table 26 shows the plasma Concentration-Time data. FIGS. 6, 7 and 8 separately present the average plasma drug concentration-time curve of 10 mg/Kg, 20 mg/Kg and 30 mg/Kg 3,4′,5-trihydroxy-stilbene-3-β-D-glucoside (TSG) administrated to Beagle dogs via intravenous injection. In Table 27 lists the pharmacokinetic parameters of 10 mg/Kg, 20 mg/Kg and 30 mg/Kg single-dose TSG injected into 5 healthy adult Beagle dogs in each of the dose groups. These pharmacokinetic parameters are obtained with non compartmental modeling method.

Test results indicate that, after intravenous injection of 3,4′,5-trihydroxy-stilbene-3-β-D-glucoside (TSG), are with, the physiological disposition of TSG in healthy Beagle dogs complies with the two-compartment model. The TSG pharmacokinetic parameter—the terminal elimination half lives (t1/2) of concentration-time curves are 167.87±170.90 min, 151.05±57.32 min and 373.09±372.98 min, respectively. The AUC0˜∞ values of 10 mg/Kg, 20 mg/Kg and 30 mg/Kg dose groups are 315.42±60.82 μg·min/ml, 745.75±175.84 μg·min/ml and 1552.71±227.28 μg·min/ml, respectively. AUC has a positive correlation with administration dose, and the correlation coefficient (r) is 0.985.

### INDUSTRIAL APPLICABILITY

New use of 3,4′,5-trihydroxy-stilbene-3-β-D-glucoside is provided. 3,4′,5-trihydroxy-stilbene-3-β-D-glucoside has efficacies of anti-myocardial ischemia by intravenous injection and/or oral administration. It is advantageous that 3,4′,5-trihydroxy-stilbene-3-β-D-glucoside as anti-myocardial ischemia drug is used to prepare the medicines for treating and/or preventing ischemic heart disease.

TABLE 26
Plasma drug concentration of single dose iv of TSG at different time (μg/ml, x̅ ± s)

| | Time (min) | | | | | | | | | | | | |
|---|---|---|---|---|---|---|---|---|---|---|---|---|---|
| Dose | 0 | 5 | 10 | 20 | 40 | 60 | 90 | 120 | 180 | 240 | 300 | 360 | 480 |
| 10 | 22.35 ± | 6.84 ± | 3.88 ± | 2.17 ± | 1.45 ± | 0.93 ± | 0.52 ± | 0.35 ± | 0.30 ± | 0.24 ± | 0.17 ± 0.06 | 0.10 ± 0.04 | 0.02 |
| mg/Kg | 4.92 | 1.89 | 0.66 | 1.19 | 0.52 | 0.55 | 0.20 | 0.10 | 0.06 | 0.04 | | | 0.04 |
| 20 | 47.68 ± | 21.82 ± | 13.07 ± | 8.25 ± | 3.48 ± | 2.36 ± | 0.84 ± | 0.68 ± | 0.46 ± | 0.35 ± | 0.29 ± 0.08 | 0.20 ± 0.05 | 0.07 ± 0.06 |
| mg/Kg | 6.51 | 4.59 | 4.17 | 4.90 | 1.23 | 1.12 | 0.60 | 0.40 | 0.11 | 0.13 | | | |
| 30 | 100.72 ± | 49.29 ± | 26.10 ± | 15.21 ± | 8.02 ± | 5.01 ± | 1.67 ± | 1.08 ± | 0.62 ± | 0.50 ± | 0.33 ± 0.05 | 0.29 ± 0.05 | 0.21 ± 0.02 |
| mg/Kg | 5.85 | 19.52 | 11.10 | 7.11 | 2.95 | 1.63 | 0.66 | 0.54 | 0.30 | 0.22 | | | |

TABLE 27
pharmacokinetic parameters of single dose of TSG iv in Beagle dogs ( x̅ ± s)

| | t_{1/2} | MRT | CI | AUC0~480 | AUC0~∞ | Vss |
|---|---|---|---|---|---|---|
| Dose | (min) | (min) | (L/min) | (μg · min/ml) | (μg · min/ml) | (L) |
| 10 mg/Kg | 167.87 ± 170.90 | 140.06 ± 124.87 | 0.03 ± 0.01 | 283.05 ± 49.85 | 315.42 ± 60.82 | 4.21 ± 3.06 |
| 20 mg/Kg | 151.05 ± 57.32 | 78.08 ± 12.24 | 0.03 ± 0.01 | 718.32 ± 179.10 | 745.75 ± 175.84 | 2.24 ± 0.78 |
| 30 mg/Kg | 373.09 ± 372.98 | 184.08 ± 240.08 | 0.02 ± 0.00 | 1432.17 ± 349.97 | 1552.71 ± 227.28 | 4.25 ± 6.35 |

1. A use of 3,4′,5-trihydroxy-stilbene-3-β-D-glucoside as shown in formula I in preparation of pharmaceuticals for treating and/or preventing ischemic heart disease, wherein said ischemic heart disease refers to asymptomatic myocardial ischemia, angina, myocardial infarction, ischemic cardiomyopathy, heart failure or sudden death

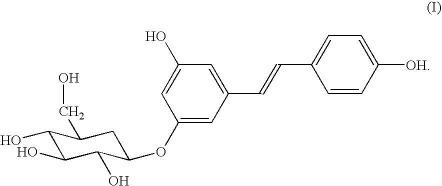

2. (canceled)

3. The use of 3,4′,5-trihydroxy-stilbene-3-β-D-glucoside according to claim 1, wherein the administrative dose of said 3,4′,5-trihydroxy-stilbene-3-β-D-glucoside is 20˜300 mg/60 kg body-weight/time.

4. The use of 3,4′,5-trihydroxy-stilbene-3-β-D-glucoside according to claim 3, wherein the administrative dose of said 3,4′,5-trihydroxy-stilbene-3-β-D-glucoside is 50˜200 mg/60 kg body-weight/time.

5. The use of 3,4′,5-trihydroxy-stilbene-3-β-D-glucoside according to claim 1, wherein said 3,4′,5-trihydroxy-stilbene-3-β-D-glucoside is used in form of preparations of oral administration or intravenous administration.

6. A use of pharmaceutical composition containing 3,4′,5-trihydroxy-stilbene-3-β-D-glucoside in preparation of pharmaceutical to treat and/or prevent ischemic heart disease.

7. The use of pharmaceutical composition according to claim 6, wherein every unit of the preparation of the said pharmaceutical composition contains 20˜300 mg of 3,4′,5-trihydroxy-stilbene-3-β-D-glucoside and pharmaceutically acceptable adjuvant, where said every unit of preparation refers to a total amount of preparation needed in a single dose of administration.

8. The use of pharmaceutical composition according to claim 7, wherein every unit of the preparation of said pharmaceutical composition contains 50˜200 mg of 3,4′,5-trihydroxy-stilbene-3-β-D-glucoside with pharmaceutically acceptable adjuvant.

9. The use of pharmaceutical composition according to claim 6, wherein said pharmaceutical composition is in form of oral administration or intravenous administration.

10. A method for treating ischemic heart disease comprising administering a subject an effective amount of 3,4′,5-trihydroxy-stilbene-3-β-D-glucoside as shown in formula I

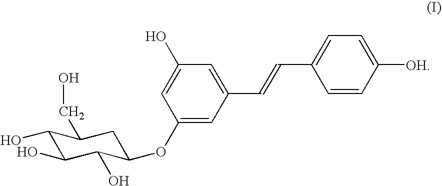

11. The method of claim 10, wherein said ischemic heart disease is selected from the group consisting of asymptomatic myocardial ischemia, angina, myocardial infarction, ischemic cardiomyopathy, heart failure or sudden death.
